(19) United States
(12) Patent Application Publication
(10) Pub. No.: US 2011/0015084 A1
(43) Pub. Date: Jan. 20, 2011

(54) Methods for Identifying Genetic Linkage

(75) Inventors: Allen Christian, St. Louis, MO (US); Michael W. Petersen, St. Louis, MO (US); Xudong Ye, St. Louis, MO (US); David Alan Somers, St. Louis, MO (US); Jennifer Anne Rinehart, St. Louis, MO (US); LaRee Witte Frank, St. Louis, MO (US); Shengzhi Pang, St. Louis, MO (US); Amy Marie Nichols, St. Louis, MO (US); John Alan Korte, St. Louis, MO (US); Heping Yang, Chesterfield, MO (US)

Correspondence Address: THOMPSON COBURN, LLP, ONE US BANK PLAZA, ST. LOUIS, MO, 63101, US

(73) Assignee: MONSANTO TECHNOLOGY LLC, St. Louis, MO (US)

(21) Appl. No.: 12/739,683

(22) PCT Filed: Oct. 23, 2008
(86) PCT No.: PCT/US08/80997

§ 371 Date: Sep. 28, 2010

Related U.S. Application Data
(60) Provisional application No. 60/982,615, filed on Oct. 25, 2007.

(51) Int. Cl. C40B 30/04; G01N 33/50; C12Q 1/68; G06F 19/00

(52) U.S. Cl. 506/9; 436/94; 435/6; 702/19

(57) ABSTRACT

The present invention provides a high-throughput system for determining linkage of distinct polynucleotides and determining the sequence of polynucleotides that are linked to the distinct polynucleotides. The methods are particularly useful for analyzing transgenes in a transformed host organism. The disclosed methods provide for the detection of linkage between distinct transgenic polynucleotides in transformed hosts and sequencing of DNA regions linked to the distinct transgenic polynucleotides. Methods for identifying a transgenic plant containing a transgene insertion in an undesirable genomic location are also disclosed.

### CROSS-REFERENCE TO RELATED APPLICATIONS

This application claims the benefit of U.S. Provisional Patent Application Ser. No. 60/982,615, filed on Oct. 25, 2007 and incorporated herein by reference in its entirety.

### STATEMENT REGARDING FEDERALLY SPONSORED RESEARCH OR DEVELOPMENT

Not Applicable.

### APPENDIX

Not Applicable.

### BACKGROUND OF THE INVENTION

One of the goals of plant genetic engineering is to produce plants with agronomically desirable characteristics or traits. The proper expression of a desirable transgene in a transgenic plant is one way to achieve this goal. Progress in molecular biology has enabled the seemingly routine insertion of foreign genes into plants, animals and microorganisms, usually with the intention of conferring desirable traits in the receiving (host) organism. For example, a gene of interest which encodes a protein relating to a specific trait in one species may be introduced into another species. In a successful transformation, enzymes in the host organism use the foreign gene which is made up of a DNA sequence as a template to synthesize a single stranded messenger nucleic acid molecule (mRNA) chain which serves as a code that is read by other cellular factors to produce a new protein in a process called translation. The new protein may cause the host organism to exhibit a new trait, such as herbicide tolerance (U.S. Pat. No. 4,940,835, herein incorporated by reference in its entirety).

Any known method used of transforming plants (for example, Agrobacterium-mediated transformation, U.S. Pat. No. 6,603,061, herein incorporated by reference in its entirety) has the potential to incorporate two or more gene fragments that may or may not be on the same chromosome or loci when inserted into the plant genome. When these fragments are on different loci they can segregate from each other in subsequent generations. This phenomenon can be both useful and detrimental depending on the goal. If the goal is to produce a stable plant that has two or more genes that are required to give a specific phenotype, then it is important that they be on the same locus to prevent loss of 1 or more of the genes in subsequent progeny. If the goal is to have a plant that can free itself from a gene that is not wanted in the progeny (i.e. and selectable marker gene), then it is imperative that this gene be inserted on a separate locus.

Plants transformed with multiple genes for eventual commercial application generally have at least two genes inserted into them during transformation (the product of which will be an R0 plant). The first is the gene of interest, or GOI, which is the gene encoding the trait that is desired in the plant. The second is a selectable marker (which enables a plant to grow under a condition in which it normally would not); this gene is used during R0 plant growth to separate those plants that received transgenic material from those that did not. For initial selection, it is therefore important that each plant have at least one copy of the marker gene. Single copy, selectable marker free transgenic plants are ideal for trait commercialization. Thus, it is important that the insertion sites for the GOI and the marker genes be far enough apart, physically, within the plant's genome that they can be segregated away from one another during a plant breeding process. The production of a marker-free transgenic plant is thus determined by the two separate DNA linkage patterns. If they (GOI and marker gene) are integrated together in same genomic locus, they are linked and transmitted to progeny together. Only when the two DNAs are inserted into different chromosomes or unlinked locus, can a marker free plant be produced by segregation in progeny. The generation and selection of high quality commercial events are thus dependent on delivery of 2 unlinked DNA fragments, one containing GOI traits, the other the selectable marker. If greenhouse and field space were unlimited, GOI/marker linkage could easily be determined at an R1 stage by techniques known in the art (such as by Invader or PCR analysis). However, as space is currently limited, it is highly desirable to have a technique to determine if unlinked genes of interest are present in a plant. Until now, assessing whether the GOI and the marker are linked requires a time-consuming molecular characterization process using Southern hybridization (J Mol Biol., 98:503-517.).

Single copy, selectable marker free transgenic plants are ideal for trait commercialization. The generation and selection of these high quality events are mostly dependent on delivery of 2 unlinked DNA fragments, one containing GOI traits, the other selectable marker, for example by gene gun bombardment (as illustrated in U.S. Pat. No. 5,015,580; U.S. Pat. No. 5,550,318; U.S. Pat. No. 5,538,880; U.S. Pat. No. 6,160,208; U.S. Pat. No. 6,399,861; and U.S. Pat. No. 6,403,865, all of which are herein incorporated by reference) or Agrobacterium-mediated 2T-DNA transformation (described in U.S. Patent Application Publication No. US20030110532, herein incorporated by reference in its entirety). Assessing whether the GOI and the marker are linked requires a time-consuming molecular characterization process using Southern hybridization. The production of a marker free transgenic plant is determined by the two separate DNA linkage patterns. If they (GOI and marker gene) are integrated together in same genomic locus, they are linked and transmitted to progeny together. Only when the two DNAs are inserted into different chromosomes or unlinked locus, can a marker free plant be produced by segregation in progeny. There are several problems with making early linkage predictions using gel based Southern blot analysis; it is labor intensive, and cannot be easily automated. An automatable method of high-throughput linkage mapping is necessary to optimize 2T analyses.

PCR and Southern hybridization are routine tools for analysis of transgenes, but each method is highly dependent on manual manipulation, is time-consuming, cost ineffective and difficult to adapt automation. The standard methods in the art for determining transgene linkage are either to use a Southern blot analysis on the R0 generation, or to test the R1 progeny for segregation. Southern analysis establishes linkage by doing two things. First, it separates the DNA into populations (bands, in this case) by running it on an agarose gel, in which smaller fragments run more rapidly than larger fragments, resulting in a length-based separation. Next, it allows visualization of the desired fragments by oligonucleotide probing. The Southern method is low throughput and requires extensive labor to perform, while testing the progeny costs valuable time and greenhouse space. Therefore, there is a need in the art for a simple screening method to detect linkages of transgenes, which can be easily adapted to a high-throughput automatable process.

Biotinylation is widely used to enable isolation, separation, concentration and further downstream processing and analysis of biomolecules (for example, methods described in U.S. Pat. No. 5,948,624, U.S. Pat. No. 5,972,693, and U.S. Pat. No. 5,512,439, all of which are herein incorporated by reference in their entireties). There are a variety of commercially available biotinylation reagents that target different functional groups like primary amines, sulfhydryls, carboxyls, carbohydrates, tyrosine and histidine side chains and guanidine and cytosine bases. The use of short, sequence-specific oligonucleotides functionalized with biotin (or the equivalent, e.g. digoxigenin) and magnetic beads to separate specific DNA sequences from the genome for subsequent analysis has multiple uses. In each case, the goal is to enrich the population of DNA for a particular sequence, allowing subsequent analysis to be carried out that could not be done in the presence of the entire genomic complement of DNA. Such bead-based methods are especially suited to high-throughput and automation. A novel method that utilizes bead-based isolation for the analyzing characteristics of transgenic organisms would fill a need in the art.

Methods in the art that attempt to facilitate transgene linkage analysis include haplotype mapping (described in Nucleic Acids Res. 1993 Jan. 11; 21(1):13-20, herein incorporated by reference), which uses haploid equivalents of DNA and the polymerase chain reaction. The approach is analogous to classical linkage mapping; two essential elements are replaced by in vitro analogues: chromosome breakage and segregation. DNA from any source is broken randomly by gamma-irradiation or shearing. Markers are then segregated by diluting the resulting fragments to give aliquots containing approximately 1 haploid genome equivalent. Linked markers tend to be found together in an aliquot. After detecting markers using the polymerase chain reaction, map order and distance can be deduced from the frequency with which markers ‘co-segregate’. Other methods include that are described in U.S. Patent Application Publication No. US20040058334, which provides for genetic linkage analysis comprising inducing mitotic recombination in a parental cell line. None of the known methods in the art provide for a rapid, efficient, cost-effective, robust, high-throughput analysis of transgene linkage.

### SUMMARY OF THE INVENTION

It is in view of the above problems that the present invention was developed.

The invention provides methods for determining linkage of at least two distinct polynucleotides, that comprise the steps of obtaining a sample comprising at least two distinct polynucleotides, isolating or identifying a first distinct polynucleotide within the sample, where isolation or identification is not effected in a gel matrix by electrophoresis, determining a measurable feature of the first distinct polynucleotide of the previous step, comparing the measurable feature from the previous step to a measurable feature of a second distinct polynucleotide sequence; and calculating a relationship between the first and the second distinct polynucleotides, wherein the results of the relationship are used to determine the linkage status of the two distinct polynucleotides. In practicing this method, the isolation of the first distinct polynucleotide segment can be effected in solution. In certain embodiments of this method, the measurable feature of the first or second distinct polynucleotide can be selected from the group consisting of a Cycle Threshold (CT) value, a molecular weight of a defined sequence of the polynucleotides, a fluorescence value, a sample mass, a molarity and a polynucleotide sequence. In other embodiments of this method, the measurable feature can be obtained by a method selected from the group consisting of a symmetric polymerase chain reaction (PCR) assay, an asymmetric polymerase chain reaction (PCR) assay, quantitative RT-PCR assay, fluorescence spectroscopy assay, a hybridization assay and sequencing. In certain embodiments, calculating said relationship can further comprise the step of normalizing ratio values for the copy number of the first and the second distinct polynucleotide sequences.

The invention also provides methods for determining linkage of at least two distinct polynucleotides in a sample, comprising the steps of obtaining a sample comprising one or more distinct polynucleotides, hybridizing a first probe and a second probe to the polynucleotides in the sample from the previous step to obtain a hybridized polynucleotide complex; separating the hybridized polynucleotide complex from the sample, determining a measurable feature of the first distinct polynucleotide and a second distinct polynucleotide in the hybridized polynucleotide complex obtained in the previous step, comparing the measurable feature of the polynucleotide molecules hybridized to the first probe and to the second probe; and calculating a relationship between the measurable features determined in the previous step to determine linkage of the first and the second distinct polynucleotide sequences. In this method, any of the probes can be an oligonucleotide. The oligonucleotide(s) used in the method can be coupled to biotin. In other embodiments of the method, at least one of the probes can be immobilized on a solid support. The solid support can be selected from the group consisting of a bead, a filter, a column, an array and a microtiter well. When the solid support is a bead, the bead is of a type selected from the group consisting of: magnetized, dye labelled, linked to a hapten, linked to a ligand, and combinations thereof. In this method, separation can be effected by a technique selected from the group consisting of a magnetic separation, bead sorting, electrophoretic separation, and buffer exchange, or any combination thereof. In certain embodiments of this method, the measurable feature of the first or second distinct polynucleotide can be selected from the group consisting of a Cycle Threshold (CT) value, a molecular weight of a defined sequence of the polynucleotides, a fluorescence value, a sample mass, a molarity and a polynucleotide sequence. In other embodiments of this method, the measurable feature can be obtained by a method selected from the group consisting of a symmetric polymerase chain reaction (PCR) assay, an asymmetric polymerase chain reaction (PCR) assay, quantitative RT-PCR assay, fluorescence spectroscopy assay, a hybridization assay and sequencing. In certain embodiments, the solid support is pretreated with a blocking agent prior to being used to isolate a distinct polynucleotide. In certain embodiments of the methods described herein, the blocking agent used to pretreat the solid support can be a proteinaceous blocking agent. Proteinaceous blocking agents used in any of the methods described herein can comprise casein, bovine serum albumin, gelatin, or any combination thereof. In certain embodiments, calculating said relationship can further comprise the step of normalizing ratio values for the copy number of the first and the second distinct polynucleotide sequences.

In these aforementioned methods of the invention, the relationship can be a ratio of measurable features. This ratio can be determined by a sequence specific polynucleotide quantitation technique. Sequence specific polynucleotide quantitation techniques used in the method can include those effected with a hybridization probe, those effected with a quantitative mass spectrometry based technique, those effected with a quantitative polynucleotide amplification technique, and/or those effected with a quantitative polynucleotide amplification technique that comprises detection of labeled oligonucleotide probe binding or detection of dye binding. In certain embodiments of the methods, the calculations of the last step provide a ratio of the measurable features, wherein a ratio of about 1 part of the first polynucleotide sequence to about 1 part of the second polynucleotide sequence in the separated sample indicates that the two polynucleotide samples are linked. In other embodiments of the methods, the calculations of the last step provide a ratio of the measurable features, wherein a ratio of about 1 part of the first polynucleotide sequence to less than about 1 part of the second polynucleotide sequence in the separated sample indicates that the two polynucleotide samples are unlinked. In these methods, the measurable feature can be a molecular weight or mass, and wherein in assessment of an identical molar ratio for the distinct polynucleotide molecules hybridized to the first probe and to the second probe indicates that the two distinct polynucleotides are linked. These methods also include embodiments wherein evaluation of the ratio further comprises the step of normalizing ratio values for the copy number of the first and the second distinct polynucleotide sequences.

The invention further provides methods for determining linkage of at least two distinct polynucleotides, comprising the steps of obtaining a sample comprising one or more distinct polynucleotides, capturing a first distinct polynucleotide, separating the first distinct polynucleotide captured in the previous step from a polynucleotide that is not captured in the previous step to obtain a second sample of polynucleotide that comprises the polynucleotide that is not captured, capturing a second distinct polynucleotide from the second polynucleotide sample in the previous step, separating the second distinct polynucleotide captured in the previous step from polynucleotide that is not captured in the previous step to obtain an enriched sample of the second distinct polynucleotide, determining a ratio of the first distinct polynucleotide to a second distinct polynucleotide in the enriched sample of the previous step, and evaluating the ratio determined in the previous step to determine linkage of the first and the second distinct polynucleotides, wherein a ratio of about 1 part of the first distinct polynucleotide to about 1 part of the second distinct polynucleotide in the separated sample indicates that the two polynucleotides are linked. In this method, separation can be effected by a technique selected from the group consisting of a magnetic separation, bead sorting, electrophoretic separation, and buffer exchange, or any combination thereof.

Additional methods of the invention include a method of sequencing an isolated polynucleotide molecule, the method comprising the steps of obtaining a sample comprising one or more polynucleotides, isolating a first distinct polynucleotide within the sample, where the isolation can be done in a manner that is not effected in a gel matrix by electrophoresis, amplifying the first distinct polynucleotide from the previous step, and sequencing the first distinct polynucleotide. In practicing this method, the isolation of the first distinct polynucleotide segment can be effected in solution.

In practicing any of the aforementioned methods of this invention, any of the polynucleotides can be obtained from a transgenic organism. In any of the methods of this invention, the sample can be genomic DNA and the transgenic organism can be a transgenic plant. Transgenic plants analyzed by any of the methods of this invention can be selected from the group consisting of barley, corn, oat, sorghum, turf grass, sugarcane, wheat, alfalfa, banana, broccoli, bean, cabbage, canola, carrot, cassava, cauliflower, celery, citrus, cotton, a cucurbit, eucalyptus, flax, garlic, grape, onion, lettuce, pea, peanut, pepper, potato, poplar, pine, rye, rice, sunflower, safflower, soybean, strawberry, sugar beet, sweet potato, tobacco, tomato, ornamental, shrub, nut, millet, and pasture grass. In any of the methods of the invention, the first or the second distinct polynucleotide can be a transgenic polynucleotide of agronomic interest. In other embodiments, the first or the second distinct polynucleotide sequence can be a polynucleotide that encodes or is operably linked to a selectable or scoreable marker gene.

In practicing any of the aforementioned methods of this invention, any of the polynucleotides can be obtained from a non-transgenic organism. In any of the methods of this invention, the sample can be genomic DNA and the non-transgenic organism can be a plant. Plants analyzed by any of the methods of this invention can be selected from the group consisting of barley, corn, oat, sorghum, turf grass, sugarcane, wheat, alfalfa, banana, broccoli, bean, cabbage, canola, carrot, cassava, cauliflower, celery, citrus, cotton, a cucurbit, eucalyptus, flax, garlic, grape, onion, lettuce, pea, peanut, pepper, potato, poplar, pine, rye, rice, sunflower, safflower, soybean, strawberry, sugar beet, sweet potato, tobacco, tomato, ornamental, shrub, nut, millet, and pasture grass. In any of the methods of the invention, the first or the second distinct polynucleotide can be a native polynucleotide of agronomic interest. In other embodiments, the first or the second distinct polynucleotide sequence can be a polynucleotide that encodes a polymorphism in a native polynucleotide of agronomic interest. Polymorphisms include, but are not limited to, single nucleotide polymorphisms, insertions, deletions, inversions, and combinations thereof.

In practicing any of the aforementioned methods of this invention, the isolated distinct polynucleotide sequences can be cleaved by a method selected from the group consisting of: lysis, a sequence-specific cleavage agent, non-sequence specific cleavage agent, sonication, shear-stress, French press, UV radiation, ionizing radiation, and DNase, and any combinations thereof. In certain embodiments of the methods of the invention where a sequence specific cleavage agent can be used, the sequence specific cleavage agent does not cleave within the first distinct polynucleotide sequence. In other embodiments of the methods, the sequence specific cleavage agent does not cleave within either the first distinct polynucleotide sequence or within the second distinct polynucleotide sequence. The sequence specific cleavage agent can be selected from the group consisting of a restriction endonuclease, a homing endonuclease, and a Flap endonuclease, or any mixture thereof. In any of the methods of the invention, isolated distinct polynucleotide sequences can be isolated by a method selected from the group consisting of: lysis, heating, alcohol precipitation, salt precipitation, organic extraction, solid phase extraction, silica gel membrane extraction, CsCl gradient purification, and any combinations thereof.

A specific method of the invention for determining the linkage relationship between two or more distinct polynucleotides, comprises the steps of obtaining a sample of tissue from a transgenic plant, extracting the DNA from the tissue sample; digesting the DNA with a restriction enzyme, annealing the DNA with biotinylated oligonucleotides corresponding to at least one site within a known gene sequence or to at least one site within a known selectable or scoreable marker sequence, adding streptavidin-coated magnetizable beads to the annealing reaction, magnetizing the beads, eluting the trapped DNA, determining the PCR Cycle Threshold (C_{T}) values for the trapped sequences, comparing the C_{T} values and calculating a ratio of the values, and determining the linkage relationship between the DNA sequences, wherein a ratio of about 1 part to about 1 part of the distinct polynucleotides indicates that the two polynucleotides are linked. In certain embodiments, the streptavidin-coated magnetizable beads are pretreated with a blocking agent prior to being added to said annealing reaction.

The invention further provides kits for determining linkage of two distinct polynucleotides in a sample. The kits of the invention comprise at least one reagent that provides for capture of a first distinct polynucleotide and instructions for the use of the kit to determine linkage to a second distinct polynucleotide in a sample. In certain embodiments of the kits, the first distinct polynucleotide sequence can be a polynucleotide that encodes or is operably linked to a selectable or scoreable marker gene in the sample. In other embodiments, the kit can further comprise at least one reagent that provides for quantitation of the first distinct polynucleotide. In still other embodiments, the kit can further comprise a control polynucleotide that comprises the first distinct polynucleotide and/or a reagent for capture of the first distinct polynucleotide sequence. The kits for determining linkage of two distinct polynucleotides can also comprise instructions for performing any of the aforementioned methods of the invention for determining linkage. The kits for determining the sequence of at least one distinct polynucleotide can comprises instructions for performing any of the aforementioned methods of determining the sequences of at least one distinct polynucleotide.

Also provided herein are methods for determining the sequence of a linked genomic polynucleotide, where the methods comprise the steps of a) cleaving a genomic DNA sample; b) isolating a distinct polynucleotide from the cleaved sample of step (a); c) amplifying the isolated polynucleotide from step (b); and d) sequencing the amplified polynucleotides from step (c), thereby determining the sequence of a linked genomic polynucleotide. In certain embodiments of the method, the genomic DNA sample is cleaved with a sequence-specific cleavage agent. In certain embodiments, isolation can be effected with a hybridization probe. In certain embodiments, the hybridization probe can be affixed to a solid support. In these methods, the solid support can be a bead is of a type selected from the group consisting of: magnetized, dye labelled, linked to a hapten, linked to a ligand, and combinations thereof. In certain embodiments, isolation can be effected by a technique selected from the group consisting of a magnetic separation, bead sorting, electrophoretic separation, and buffer exchange, or any combination thereof. In certain embodiments, the solid support is pretreated with a blocking agent prior to being used to isolate the distinct polynucleotide in step (b). When used, the blocking agent can be a proteinaceous blocking agent. Proteinaceous blocking agents used in the methods can comprise casein, bovine serum albumin, gelatin, or any combination thereof. In certain embodiments, amplification comprises a sequence independent amplification technique. When used in these methods, sequence independent amplification technique can comprise use of random primers. In certain embodiments, amplification comprises a rolling circle amplification method. In certain embodiments, amplification comprises use of a sequence specific primer with a DNA polymerase. In certain embodiments, sequencing can be effected by pyrosequencing.

Also provided herein is a method for identifying a transgenic plant containing a transgene insertion in an undesirable genomic location, comprising the step of identifying a transgenic plant wherein a transgene has inserted into a genomic region comprising one or more retrotransposon sequences, thereby identifying a transgenic plant containing a transgene insertion in an undesirable genomic location. In these methods, the transgenic plant can be a dicot plant or a monocot plant. In certain embodiments, the retrotransposon is a TY3/gypsy-like retrotransposon. In certain embodiments, the methods can further comprise the step of culling the transgenic plant wherein a transgene has inserted into a genomic region comprising one or more retrotransposon sequences. In certain embodiments of the method, the transgene insertion that is in an undesirable location is adjacent to a retrotransposon or is within a retrotransposon. In certain embodiments, the transgene can comprise a gene used to suppress expression of an endogenous gene of the plant.

### BRIEF DESCRIPTION OF THE DRAWINGS

The accompanying drawings, which are incorporated in and form a part of the specification, illustrate the embodiments of the present invention and together with the description, serve to explain the principles of the invention. In the drawings:

FIGS. 1-12 illustrate the sequential steps in an exemplary embodiment of the methods of the invention for determining linkage of distinct polynucleotides.

FIGS. 13-18 illustrate the sequential steps in an exemplary embodiment of another method for determining linkage of distinct polynucleotides where unlinked copies of the gene of interest are enriched by first trapping the markers, removing the untrapped supernatant containing unlinked genes of interest and then trapping the gene of interest from that supernatant.

FIGS. 19-23 illustrate the sequential steps in an exemplary embodiment of the methods of the invention for sequencing an isolated polynucleotide or for determining the sequence of a linked genomic polynucleotide. Preparation of templates for sequencing of genomic DNA regions that flank the insertion site of a transgene (in this case a T-DNA) are shown. Templates prepared as per the scheme of FIGS. 19-23 can be sequenced by a variety of methods, including but not limited to, dye-deoxy chain termination based methods.

FIG. 24 illustrates real-time PCR results obtained from the 3 indicated Arabidopsis transgenic events after purification. The vertical axis indicates numbers of delta threshold cycles (Ct) that is defined as Ct reference-Ct target. More than 1000-times enrichment of the target fragments from 3 Arabidopsis transgenic events was obtained after purification.

FIG. 25 illustrates the effects of pretreatment of streptavidin magnetic beads with 0.2% I-Block (a highly purified casein-based blocking reagent) and 0.5% SDS in PBS buffer. Using 6 traps instead of 2 could increase enrichment efficiency. The vertical axis indicates numbers of delta threshold cycles (Ct) that is defined as Ct reference-Ct target. Pretreatment of streptavidin magnetic beads with 0.2% I-Block (a highly purified casein-based blocking reagent) and 0.5% SDS in PBS buffer could considerably prevent nonspecific binding of DNA and therefore reduce background.

FIG. 26 illustrates the PCR results obtained with ten pairs of primers the 8.9 kb Xba I-fragment and undigested genomic DNA, the purified Xba I-fragment (8.9 kb), Sal I-fragment (15 kb), or the Phi29 products templates. The Sal I-fragment is longer on the 3′ end but shorter on the 5′ end than the Xba I-fragment. The PCR results indicated that the enriched fragments were intact.

FIG. 27 illustrates DNA electrophoresis analysis of enriched and amplified samples. Several micrograms of products could be generated from just a few nanograms of the targeted fragment using Phi29. Lane 1, 100 bp ladder; Lane 2, 1 kb ladder; Lanes 3˜5, purified target DNA; Lane 6˜8, amplified products. The results indicated that the enriched fragments of 9 kb and 15 kb were successfully amplified.

FIG. 28 illustrates DNA electrophorograms of fragmented target DNA fragments. Panel A, Arabidopsis thaliana_S56551-At3g21150; Panel B, Arabidopsis thaliana_S56551-SUP-miRGL1; Panel C, Arabidopsis thaliana_S56520-SUP-miRGL1; Panel D, Arabidopsis thaliana_S56518-SUP-miRGL1. The peaks in light grey are DNA marker ladders. The majority of the fragmented target DNA fragments were about 850 bp in length.

FIG. 29 illustrates DNA electrophorograms of sstDNA. Panel A, Arabidopsis thaliana S56551-At3g21150; Panel B, Arabidopsis thaliana S56551-SUP-miRGL1; Panel C, Arabidopsis thaliana_S56520-SUP-miRGL1; Panel D, Arabidopsis thaliana_S56518-SUP-miRGL1. The peaks in light grey are DNA marker ladders.

FIG. 30 illustrates sequencing read distributions on the 8.9 kb target fragment contained in At3g21150. The adapter sequence was removed from the reads before mapping. The boxes on the “Position” or X-axis indicate the gene of interest. The arrows “Position” or X-axis indicate the trap (i.e. biotinylated oligonucleotides) locations. Panel A, reads obtained using 2 traps; Panel B, reads obtained using 6 traps.

FIG. 31 illustrates the Arabidopsis genomic DNA/T-DNA junction sequence found in the At_S56518 event (SEQ ID NO:1). Residues 1-498: T-DNA sequence, residues 499-509 are rearrangement sequence (italic), and residues 510-789 are Arabidopsis genomic sequence where the underlined sequence indicates the primer location. For the genomic sequence (shown in bold) only, lowercase indicates non-coding sequence and uppercase indicates coding sequence.

FIG. 32 illustrates the Arabidopsis genomic DNA/T-DNA junction sequence found in the At_S56520 event (SEQ ID NO:2). Residues 1-436 are the T-DNA sequence and residues 437 to 443 are rearrangement sequence (italic). For the Arabidopsis genomic sequence (Residues 444-1356), the underlined sequence indicates the primer location. For the genomic sequence (shown in bold) only, lowercase indicates non-coding sequence and uppercase indicates coding sequence.

FIG. 33 illustrates the Arabidopsis genomic DNA/T-DNA junction sequence found in the At_S56551 event (SEQ ID NO:3). Residues 1-201 are the T-DNA sequence and residues 201 to 1214 are the Arabidopsis genomic sequence where the underlined sequence indicates the primer location. For the genomic sequence (shown in bold) only, lowercase indicates non-coding sequence and uppercase indicates coding sequence.

FIG. 34 illustrates the confirmation of the Arabidopsis genomic DNA/T-DNA junction sequences by PCR. Lane 1, 100 bp ladder; Lane 2, At_S56518; Lane 3, At_S56520; Lane 4. At S56551.

FIG. 35 illustrates the arrangement of the transgene insertions in the Arabidopsis genome. Panel A shows the transgenic event At-S56518 where the T-DNA insertion (SUP-miRGL1) was between two typical Arabidopsis genes, AT5G11060 and AT5G11070. Panel B shows the event At-S56520 where the T-DNA insertion (SUP-miRGL1) was in a retrotransposon (AT4G05593 and AT4G05594). Panel C shows the event At-S56551 where the right border of the T-DNA insertion (SUP-miRGL1) was truncated and T-DNA insertion was adjacent to a retrotransposon (AT5G32345).

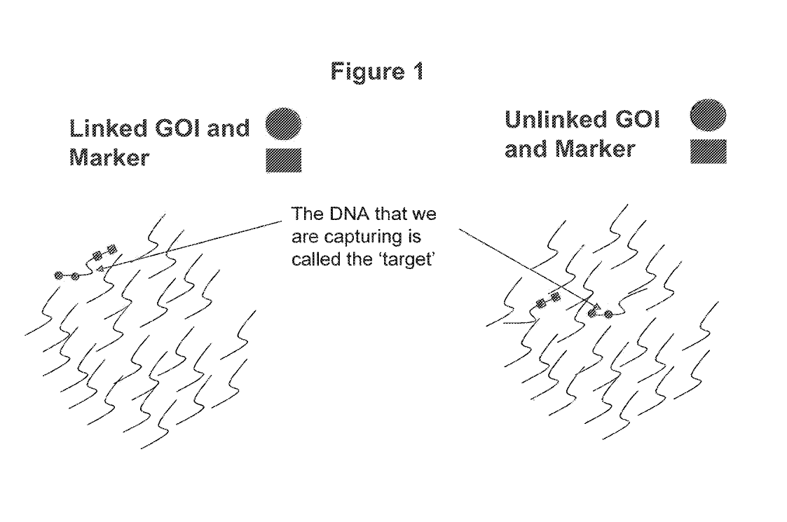

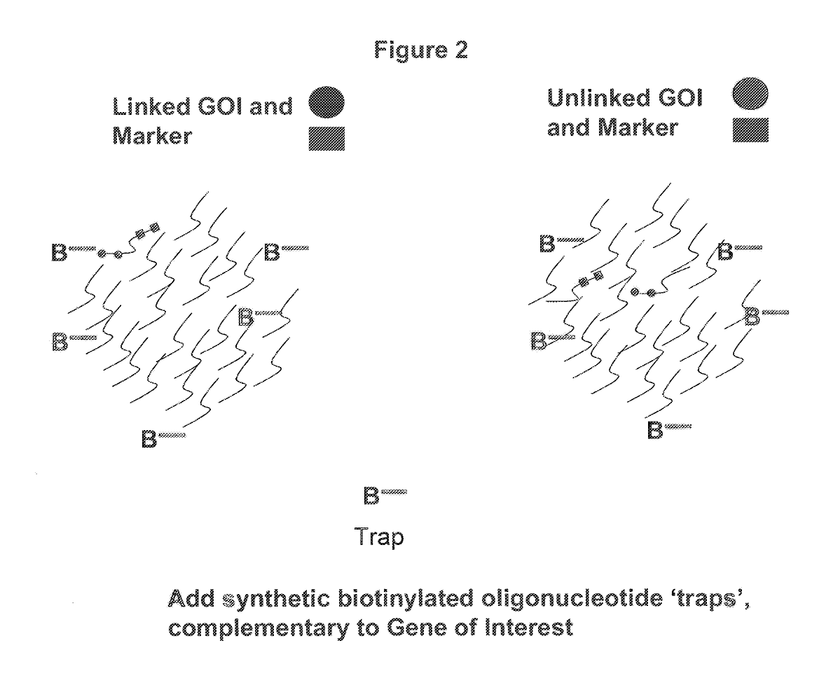

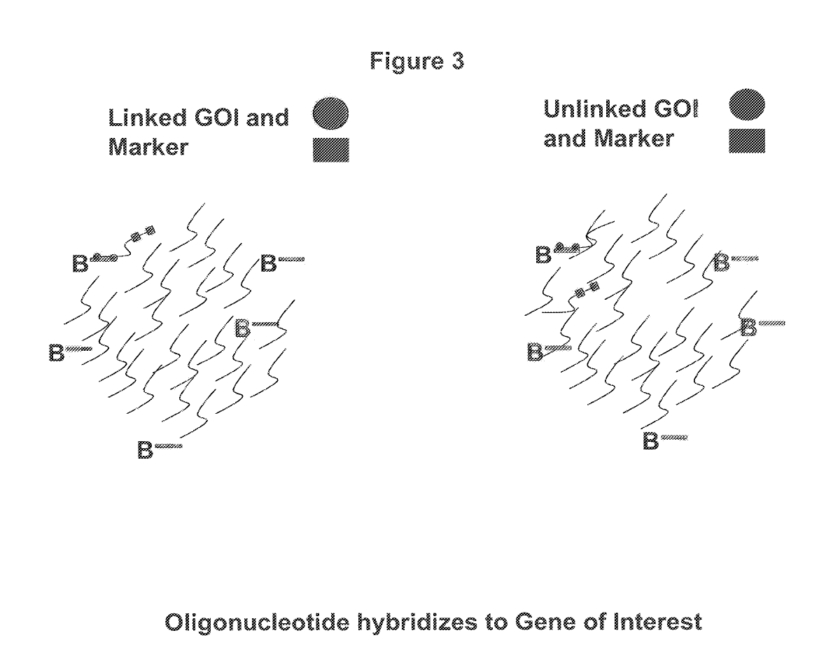

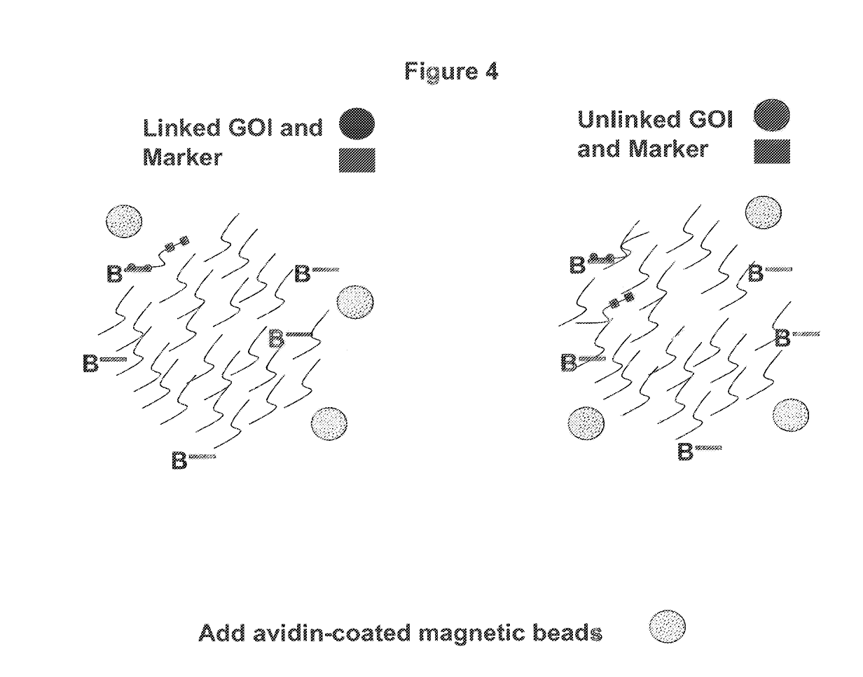

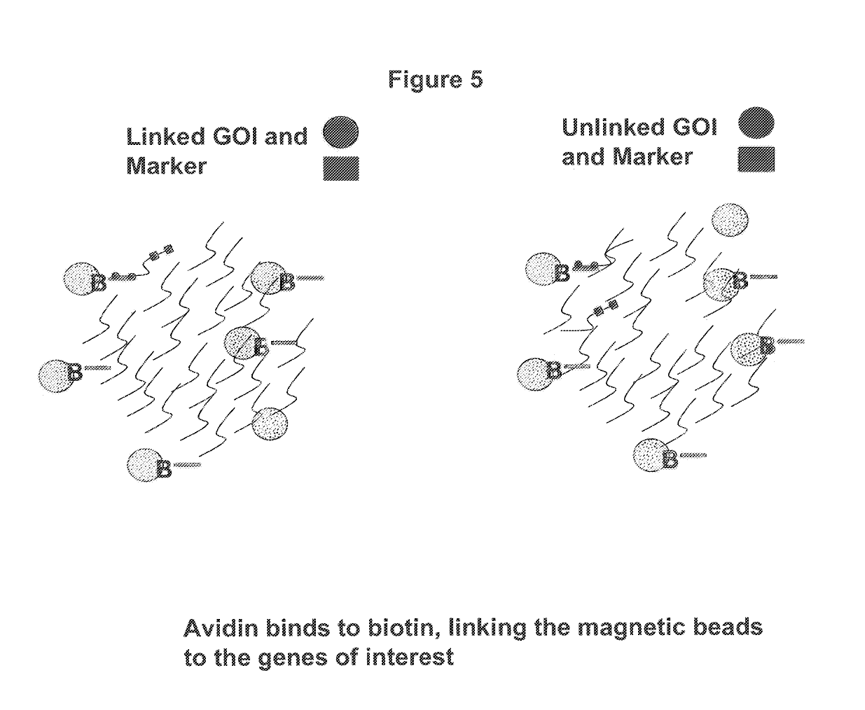

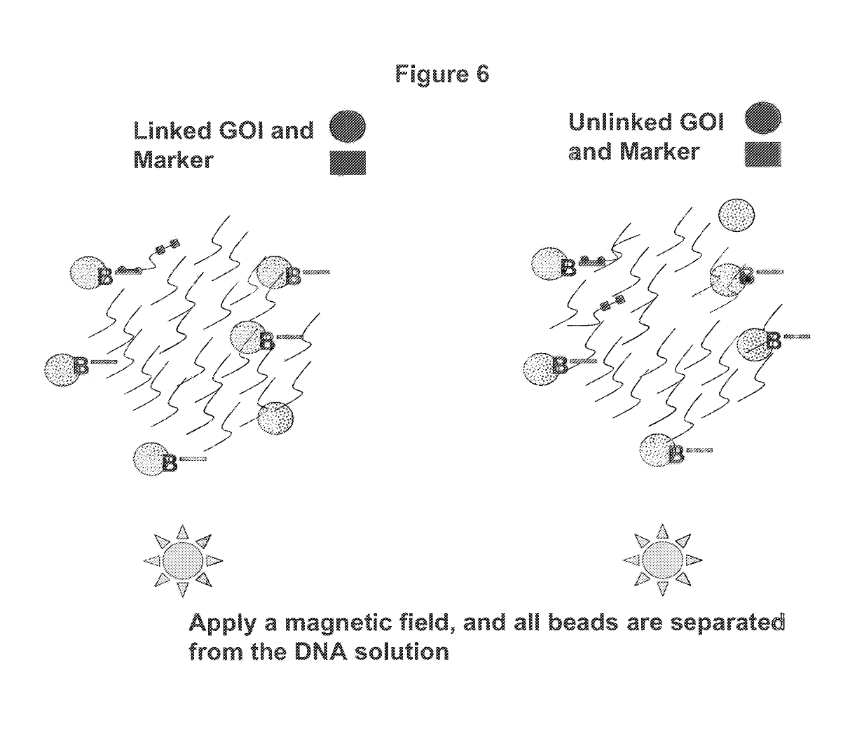

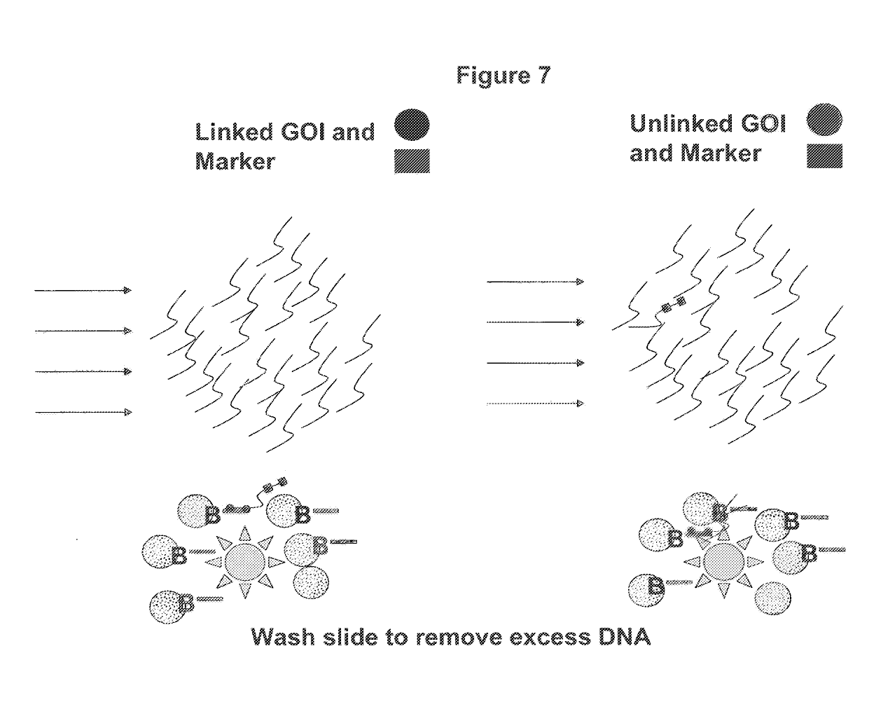

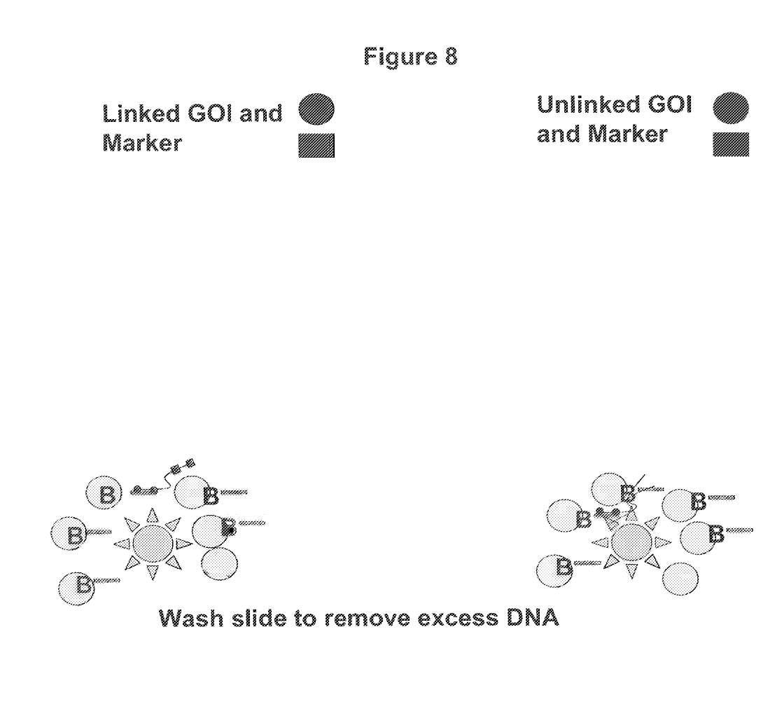

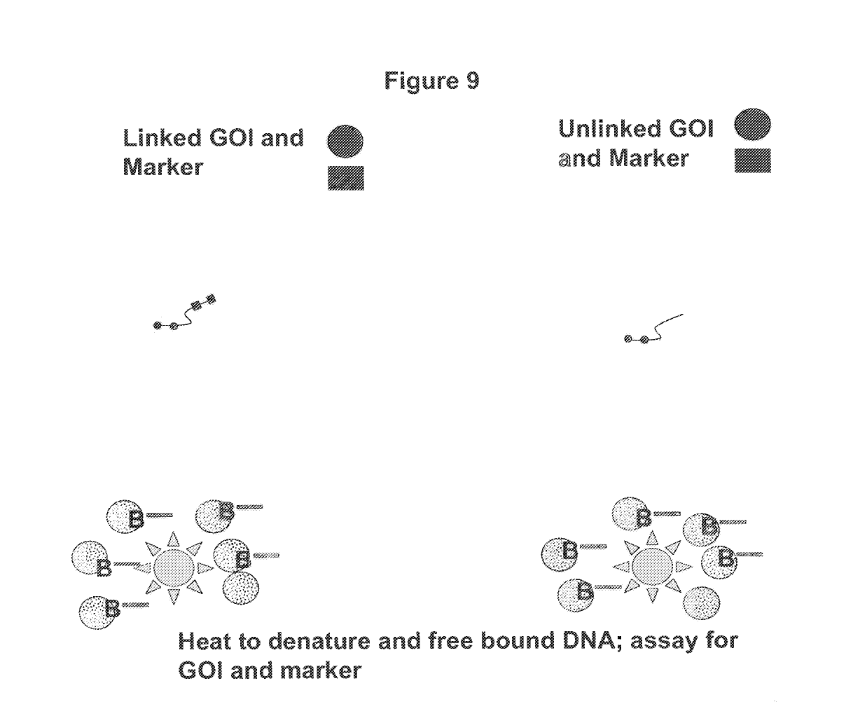

### ILLUSTRATIVE EMBODIMENTS

The definitions and methods provided define the present invention and guide those of ordinary skill in the art in the practice of the present invention. Unless otherwise noted, terms are to be understood according to conventional usage by those of ordinary skill in the relevant art. Definitions of common terms in molecular biology may also be found in Alberts et al., Molecular Biology of The Cell, 3rd Edition, Garland Publishing, Inc.: New York, 1994; Rieger et al., Glossary of Genetics: Classical and Molecular, 5th edition, Springer-Verlag: New York, 1991; and Lewin, Genes V, Oxford University Press: New York, 1994. The nomenclature for DNA bases as set forth by 37 CFR §1.822 is used. As used herein certain terms and phrases are defined as follows.

### I. DEFINITIONS

As used herein, the term “comprising”, means “including but not limited to”.

The term “Construct”, as used herein refers to any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e., operably linked.

As used herein, the phrase “distinct polynucleotide”, refers to a polynucleotide of at least 10 nucleotides in length wherein the sequence of the polynucleotide has at least one nucleotide difference relative to other polynucleotides of equal length. Two distinct polynucleotides can form a portion of either a single contiguous polynucleotide fragment (i.e. can reside on a single fragment) or can each form a portion of two separate polynucleotide fragments (i.e. can reside on two separate fragments).

The phrase “DNA construct”, as used herein refers to any DNA molecule in which two or more ordinarily distinct DNA sequences have been covalently linked. Examples of DNA constructs include but are not limited to, plasmids, cosmids, viruses, BACs (bacterial artificial chromosome), YACs (yeast artificial chromosome), plant minichromosomes, autonomously replicating sequences, phage, or linear or circular single-stranded or double-stranded DNA sequences, derived from any source, that are capable of genomic integration or autonomous replication. DNA constructs can be assembled by a variety of methods including, but not limited to, recombinant DNA techniques, DNA synthesis techniques, PCR (Polymerase Chain Reaction) techniques, or any combination of techniques.

The phrase “gene of interest”, as used herein, includes any gene that confers a desirable trait when expressed in the host organism. Genes of interest are understood to comprise both genes that encode proteins that confer a desirable trait as well as genes that provide for modulating the expression of other genes within a host to confer a desirable trait.

As used herein, “genetic marker”, means polymorphic nucleic acid sequence or nucleic acid feature.

As used herein, the term “haplotype”, means a chromosomal region within a haplotype window defined by at least one polymorphic genetic marker. The unique genetic marker fingerprint combinations in each haplotype window define individual haplotypes for that window. Further, changes in a haplotype, brought about by recombination for example, may result in the modification of a haplotype so that it comprises only a portion of the original (parental) haplotype operably linked to the trait, for example, via physical linkage to a gene, QTL, or transgene. Any such change in a haplotype would be included in our definition of what constitutes a haplotype so long as the functional integrity of that genomic region is unchanged or improved.

As used herein, the term “haplotype window”, means a chromosomal region that is established by statistical analyses known to those of skill in the art and is in linkage disequilibrium. Thus, identity by state between two inbred individuals (or two gametes) at one or more loci located within this region is taken as evidence of identity-by-descent of the entire region. Each haplotype window includes at least one polymorphic genetic marker. Haplotype windows can be mapped along each chromosome in the genome. Haplotype windows are not fixed per se and, given the ever-increasing density of genetic markers, this invention anticipates the number and size of haplotype windows to evolve, with the number of windows increasing and their respective sizes decreasing, thus resulting in an ever-increasing degree of confidence in ascertaining identity by descent based on the identity by state at the genetic marker loci.

The phrase “a heterologous promoter”, as used herein in the context of a DNA construct, refers to either: i) a promoter that is derived from a source distinct from the operably linked structural gene or ii) a promoter derived from the same source as the operably linked structural gene, where the promoter's sequence is modified from its original form.

As used herein, the phrase “high stringency hybridization conditions”, refers to nucleic acid hybridization conditions comprising a salt concentration of about 1×SSC, a detergent concentration of about 0.1% SDS, and a temperature of about 50° C., or equivalents thereof.

As used herein, the phrase “hybridized polynucleotide complex”, refers to an entity comprising two polynucleotides that are complementary over some portion of their length, wherein the two polynucleotides are hybridized to one another over that portion of their length.

As used herein, the term “linkage”, refers to the presence of two distinct polynucleotide sequences on a single chromosome.

As used herein, the term “linked”, when used in the context of two distinct polynucleotides, refers to the presence of those two distinct polynucleotide sequences on a single chromosome. It thus follows from this definition that two distinct polynucleotides located within 100 centiMorgans, 50 centiMorgans, 20 centiMorgans, 10 centiMorgans, 5 centiMorgans, or 1 centiMorgan or less of one another on a single chromosome are also linked.

As used herein, the term “measurable feature”, refers to a qualitative or quantitative observation of a distinct polynucleotide sequence. For example, a measurable feature of a polynucleotide molecule may include, but is not limited to, sequence, structure, spectroscopic properties, molecular weight, mass, molarity, electrophoretic mobility, cycle threshold (CT) value, hybridization temperature melting point (Tₘ), melting point, density, pH, pK, pI, composition, size, solubility, reactivity, stability, and/or radioactivity.

As used herein, the term “native polynucleotide”, refers to an endogenous polynucleotide of a non-transgenic organism. This endogenous polynucleotide may be polymorphic.

As used herein, the term “not effected by”, means that a given action is not done or accomplished by a stated procedure.

The phrase “operably linked”, as used herein refers to the joining of nucleic acid sequences such that one sequence can provide a required function to a linked sequence. In the context of a promoter, “operably linked” means that the promoter is connected to a sequence of interest such that the transcription of that sequence of interest is controlled and regulated by that promoter. When the sequence of interest encodes a protein and when expression of that protein is desired, “operably linked” means that the promoter is linked to the sequence in such a way that the resulting transcript will be efficiently translated. Nucleic acid sequences that can be operably linked include, but are not limited to, sequences that provide gene expression functions (i.e., gene expression elements such as promoters, 5′ untranslated regions, introns, protein coding regions, 3′ untranslated regions, polyadenylation sites, and/or transcriptional terminators), sequences that provide DNA transfer and/or integration functions (i.e., T-DNA border sequences, site specific recombinase recognition sites, integrase recognition sites), sequences that provide for selective functions (i.e., antibiotic resistance markers, biosynthetic genes), sequences that provide scoreable marker functions (i.e., reporter genes), sequences that facilitate in vitro or in vivo manipulations of the sequences (i.e., polylinker sequences, site specific recombination sequences) and sequences that provide replication functions (i.e., bacterial origins of replication, autonomous replication sequences, centromeric sequences).

As used herein, the term “oligonucleotide”, refers to a polymer comprising at least three and no more than about 300 covalently linked nucleotides.

As used herein, “polymorphism”, means the presence of one or more variations of a nucleic acid sequence at one or more loci in a population of one or more individuals. The variation may comprise but is not limited to, one or more base changes, the insertion of one or more nucleotides or the deletion of one or more nucleotides. A polymorphism includes a single nucleotide polymorphism (SNP), a simple sequence repeat (SSR) and indels, which are insertions and deletions. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation.

As used herein, the term “polynucleotide”, refers to a polymer comprising at least two covalently linked nucleotides.

As used herein, the term “ratio”, refers to the relative magnitudes of quantities of one particular measurable feature of two distinct polynucleotides in a sample. A ratio is a quantitative expression of the relationship between two measurable features. For example, the CT value of one polynucleotide may be compared to the CT value of a second polynucleotide, and a ratio of values determined. It is understood that a given sample may contain more than two (2) distinct polynucleotides. In such cases, more than one ratio between different pairs of distinct polynucleotides in the sample can be determined. For example, in cases where a given sample contains distinct polynucleotides A, B, C, and D, it is possible to determine ratios for A:B, A:C, A:D, and all other combinations thereof.

As used herein, the term “relationship”, refers to the results of a correlation between the measurable features of two or more distinct polynucleotides in a sample. A relationship does not necessarily indicate an association, but instead is an objective observation and comparison of the features of two or more samples.

As used herein, the term “R0”, refers to any plant regenerated through tissue culture, including a transgenic plant.

As used herein, the term “R1”, refers to the first progeny of a cross between R0 parents, including one or more transgenic parents.

As used herein, the term “F1”, refers to the first generation of a cross between normal i.e., non-transgenic or wild type parents.

As used herein, the phrases or terms “sequence identity”, “sequence similarity”, or “homology”, is used to describe sequence relationships between two or more nucleotide sequences. The percentage of “sequence identity” between two sequences is determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa. A first nucleotide sequence when observed in the 5′ to 3′ direction is said to be a “complement” of, or complementary to, a second or reference nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence.

As used herein, the term “single nucleotide polymorphism”, also referred to by the abbreviation “SNP”, means a polymorphism at a single site wherein said polymorphism constitutes a single base pair change, an insertion of one or more base pairs, or a deletion of one or more base pairs.

As used herein, the term “solid support”, refers to a matrix to which a molecule of any sort may be attached. Preferably, a solid support is an insoluble material to which a molecule may be attached so that said molecule may be readily separated from other components in a reaction. A solid support may include, but is not limited to, a filter, a chromatography resin a bead, a magnetic particle, or compositions that comprise glass, plastic, metal, one or more polymers and combinations thereof.

As used herein, the term “substantially homologous”, or “substantial homology”, with reference to a nucleic acid or polypeptide sequence, refers to a nucleotide or polypeptide sequence that has about 65% to about 70% sequence identity, or more preferably from about 80% to about 85% sequence identity, or most preferable from about 90% to about 95% sequence identity, to about 99% or 100% sequence identity, with another nucleotide or polypeptide sequence.

The term “transformation”, as used herein refers to a process of introducing an exogenous DNA sequence (e.g., a vector, a recombinant DNA molecule) into a cell or protoplast in which that exogenous DNA is incorporated into a chromosome or is capable of autonomous replication.

The phrase “transgenic plant”, refers to a plant or progeny thereof derived from a transformed plant cell or protoplast, wherein the plant DNA contains an introduced exogenous DNA molecule not originally present in a native, non-transgenic plant of the same species.

The term “vector”, as used herein refers to any recombinant polynucleotide construct that may be used for the purpose of transformation, i.e., the introduction of heterologous DNA into a host cell.

### II. METHODS OF THE INVENTION

The present invention is directed to methods of analyzing linkage of distinct polynucleotides. These particular methods of analyzing polynucleotide linkage can be used for any application or situation where it is desirable to determine if two distinct polynucleotides are linked. Examples of applications or situations where this method of determining linkage can be employed include, but are not limited to, characterization of insertion, deletion, and/or recombination events in the genome of an organism. This genome includes all of the resident genetic information in a host, such as the host chromosome, the genomes of sub-cellular organelles (i.e. mitochondrial or plastid genomes), artificial chromosomes, or extra-chromosomal elements which may be either natural or synthetic in origin. Such genomic events can be catalyzed by any one or combination of agents that include, but are not limited to, chemical mutagens, transposases, both site-specific and non-site specific recombinases, or by any mechanism by which a exogenous DNA sequence can be introduced into a host organism. Mechanisms by which an exogenous sequence can be introduced into a host organism include, but are not limited to, transformation, transfection, transduction, or conjugation. One particular and useful application of the methods disclosed herein relates to the characterization of exogenous DNA insertions (in this case transgenes) into the chromosome of a transgenic host organism. The methods of this invention can provide for transgene linkage analysis wherein it is determined if one or more transgene(s) of interest have integrated at the same genomic location as a transgene comprising a selectable or scoreable marker (i.e. are linked). The methods of this invention can also provide for sequencing or isolation of the genomic DNA adjacent to or flanking a genomic insertion (such as a transgene or transposon), genomic deletion, or genomic recombination event.

The present invention finds many applications. The present invention provides high-throughput non-Southern Blot-based linkage detection methods (i.e. methods that are not based on electrophoretic separations and transfer) which are useful for determining the linkage relationship between DNA molecules in a transformed or non-transformed organisms. Determination of linkage relationships between DNA molecules can be used in haplotype analysis (i.e. determining if a given plant from a breeding population has inherited a haplotype), developing physical maps, linkage mapping, walking towards a trait in order to clone the underlying gene or mutant gene of interest, determining linkage of genetic markers, trait stacking analysis, flanking DNA isolation/analysis, and SNP detection.

In one embodiment of the present invention, methods and compositions are provided to trap a known transgene from a background of genomic DNA, enabling the establishment of the insertion site of the transgene in the transgenic organism's genome (e.g. plant genome) by sequencing outward from the known DNA sequence, in both directions (i.e. by sequencing into the regions. This sequence (flank sequence) could be compared to known DNA sequence databases and the exact insertion position in the genome established.

In another embodiment of the present invention, methods are provided that enable targeted sequencing of specific genomic regions, using the trapping and sequencing techniques previously described. For example, the DNA sequence of a species of a given plant (e.g. soy) is established from one strain. Mapping of single nucleotide polymorphisms among different strains of the same species, useful for breeding purposes, could be done by this technique.

In other embodiments of the invention, linkage between distinct polynucleotides such as transgenes inserted into the genome of a host organism and other distinct polynucleotides that are genetic markers for haplotypes can be determined. Determination of linkage of these distinct polynucleotides by the methods of this invention is particularly useful for analysis of the T-type genomic regions as described in U.S. Patent Application Publication No. 20060282911. A T-type genomic region is a novel genetic composition comprising at least one transgene, with suitable levels of expression, in genetic linkage with a haplotype. In a preferred embodiment the linkage of a transgene with a haplotype should have no observable deleterious effect on the functional integrity of the haplotype due to the local insertion of the transgene. Additionally a haplotype of a T-type genomic region could be functionally enhanced as a result of the integration into genetic linkage of a transgene. By using the linkage methods of this invention, linkage relationships between the transgene and associated haplotypes can be tracked in breeding populations that result when germplasm comprising a transgene insertion is outcrossed to distinct germplasms.

### III. METHODS OF ANALYZING LINKAGE AND FOR SEQUENCING: SAMPLE PREPARATION, POLYNUCLEOTIDE ISOLATION, MEASUREMENT, SEQUENCING AND ANALYSIS

The methods of this instant invention can be subdivided into the steps of sample preparation, polynucleotide isolation, measurement and analysis. The methods of the invention directed to obtaining sequence data can use the same preparation and isolation steps.

III.a. Sample Preparation

The initial step of the method comprises preparation of genomic DNA or RNA from the organism that contains one or more distinct polynucleotide(s) of interest. This sample preparation can be achieved via any technique that provides the genomic DNA or RNA in a sufficiently unfragmented form and at sufficient levels of purity. The degree of fragmentation permitted is at least dependent on the size of the distinct polynucleotides to be isolated and analyzed. When the polynucleotides of interest are large (i.e. about 20-50 kB of nucleotides in length or more), techniques that provide the genomic DNA or RNA in relatively intact form (i.e. at least about 100 kB of nucleotides in length) are used. When the polynucleotides of interest are smaller (i.e. about 0.1 kB to about 20 kB), techniques that provide the genomic DNA or RNA in at least moderately sized fragments (i.e. at least about 20 to 50 kB of nucleotides in length) or in relatively intact form can be used. Furthermore, when the polynucleotides of interest are still smaller (i.e. about 10 nucleotides to about 100 nucleotides in length), techniques that provide the genomic DNA or RNA in at least small fragments (i.e. at least about 20 kb of nucleotides in length) or in relatively intact form can be used. In general, one of skill in the art will appreciate that the key feature of the method is that the genomic DNA or RNA provided by the sample preparation technique is not fragmented to a degree that would preclude detection of linkage of the distinct polynucleotides of interest.

The genomic DNA samples used in any of the methods of the invention include but are not limited to, genomic DNA isolated directly from an organism, cloned genomic DNA from the organism, or amplified genomic DNA from an organism. Amplification can be either symmetric or asymmetric. Symmetric amplification as described employs the polymerase chain reaction (PCR) (Mullis et al., 1986 Cold Spring Harbor Symp. Quant. Biol. 51:263-273; European Patent No. 50,424; European Patent No. 84,796; European Patent No. 258,017; European Patent No. 237,362; European Patent No. 201,184; U.S. Pat. No. 4,683,202; U.S. Pat. No. 4,582,788; and U.S. Pat. No. 4,683,194), using primer pairs that are capable of hybridizing to the region of interest and synthesizing overlapping complementary strands. Asymmetric amplification refers to any process whereby an unequal concentration of primers is used to generate more of one strand than another. Genomic DNA samples for use in the methods of this invention can also be obtained by repeated rounds of synthesis from a template strand by annealing one or more primers oriented in a single direction on a DNA strand, extending those annealed primers with a suitable polymerase, denaturing the extension product from the template, and repeating the process as necessary.

Given these general considerations of the average DNA or RNA fragment size in the population of interest, an appropriate method for isolation of genomic DNA or RNA from the host organism can be selected. Any method for extracting genomic DNA from tissue samples that provide genomic DNA of sufficient purity to be captured by a hybridization probe or to be amplified can be used. Genomic DNA samples can be obtained by methods including, but not limited to, lysis, heating, alcohol precipitation, salt precipitation, organic extraction, solid phase extraction, silica gel membrane extraction, CsCl gradient purification, and any combinations thereof. Examples of high throughput plant DNA isolation procedures that can be used include, but are not limited to, those described by Lange et al., “A Plant DNA Isolation Protocol Suitable for Polymerase Chain Reaction Based Marker-Assisted Breeding”, Crop Science, 38:217-220 (1998), Dilworth and Frey, “A Rapid Method for High Throughput DNA Extraction from Plant Material for PCR Amplification”, Plant Molecular Biology Reporter 18: 61-64, (2000). Commercial kits for isolating plant genomic DNA can also be employed to obtain DNA suitable for use in this method (Extract-N-Amp™ Plant PCR Kit, Sigma-Aldrich, Saint Louis, Mo., USA; MagAttract™ 96 DNA plant kit or DNAeasy™ 96 Plant kit, both from Qiagen, Inc. Valencia, Calif. USA).

III.b. Polynucleotide Isolation

In the methods of the invention, the distinct polynucleotides are isolated from other distinct polynucleotides in the sample. This can be effected with a hybridization probe that is either affixed, or is capable of being affixed, to a solid support. Once the distinct polynucleotide of interest has been captured from the sample by binding of the hybridization probe that is affixed to a solid support, the solid support can be separated from the remainder of the solution to isolate the distinct captured polynucleotide. The solid support can also be subjected to any number of buffer exchange or washing steps to further purify the distinct polynucleotide captured by the hybridization probe. Once the distinct polynucleotide has been separated from other distinct and unlinked polynucleotides, it can be released from the solid support by dissociating the polynucleotide from the hybridization probe. Dissociation can be effected by increasing the temperature, decreasing the salt concentration, or combinations thereof such that the probe and polynucleotide are no longer hybridized. The dissociated polynucleotide can then be subjected to appropriate measurement techniques in subsequent steps of the method.

Affixation of the hybridization probe to the solid support can be either through a covalent linkage or through other non-covalent interactions. Non-covalent interactions include, but are not limited to, binding interactions between a protein and a hapten. An exemplary non-covalent interaction is one where a biotin-labelled oligonucleotide is bound by a streptavidin molecule that is in turn coupled to a solid support.

Solid supports useful in the practice of these methods include, but are not limited to, a bead, a filter, a column, an array and a microtiter well. When the support is a microtiter plate, the microtiter plates can have as few as 8 wells, or as many as 24, 96, 384, 1536 or 3456 wells. The microtiter plates can be constructed from materials including, but not limited to, polystyrene, polypropylene, or cyclo-olefin plastics.

When the solid support is a bead, the bead can be magnetized, dye labelled, linked to a hapten, linked to a ligand, and combinations thereof. The use of magnetized beads is particularly useful in that such beads can be rapidly and efficiently separated from the solution by applying a magnetic field to the sample. Isolation of various types of biological macromolecules through use of magnetic particles and methods of attaching hybridization capture probes are disclosed in U.S. Pat. Nos. 5,508,164, and 5,665,582. The use of magnetic beads to isolate hybridization complexes has been described in a variety of patent (U.S. Patent Application Publication Nos 20050079510 and 20050284817) and non-patent publications (Anal Biochem. 1992 Feb. 14; 201(1):166-9; Biotechniques. 2002 June; 32(6):1296, 1298-1300; Hawkins, et al., Nucleic Acids Res. 1995 (23): 4742-4743). Magnetized beads can also be analyzed in array formats (U.S. Patent Application Publication No 20020081714). Commercial sources of magnetic beads, magnetic bead base purification kits, and apparati for effecting magnetic separations include Agencourt Biosciences (Beverly, Mass. USA), ProMega (Madison, Wis. USA), and Invitrogen (Carlsbad, Calif. USA).

Alternatively, the beads can contain a unique identifying label. In particular, beads dyed with fluorochromes that can be distinguished by their spectrophotometric or fluorometric properties can be coupled to the nucleic acid molecules for separating distinct polynucleotides from one another or from unbound polynucleotides in the solution. Such bead based systems have been described (U.S. Pat. No. 5,736,330). Dye labelled beads, analysis reagents and apparati for bead separation have also been described (U.S. Pat. Nos. 6,649,414, 6,599,331, and 6,592,822) and are available from Luminex Corporation (Austin, Tex. USA). Such bead based systems are particularly useful for applications where multiple sets of distinct polynucleotides are queried in a given sample (i.e. multiplex analyses).

As used herein, two nucleic acid molecules are said to be capable of hybridizing to one another if the two molecules are capable of forming an anti-parallel, double-stranded nucleic acid structure. A nucleic acid molecule is said to be the “complement” of another nucleic acid molecule if they exhibit “complete complementarity” i.e. each nucleotide in one sequence is complementary to its base pairing partner nucleotide in another sequence. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Nucleic acid molecules which hybridize to other nucleic acid molecules, e.g. at least under low stringency conditions are said to be “hybridizable cognates” of the other nucleic acid molecules. Conventional stringency conditions are described by Sambrook et al., Molecular Cloning, A Laboratory Manual, 2nd Ed., Cold Spring Harbor Press, Cold Spring Harbor, N.Y. (1989) and by Haymes et al., Nucleic Acid Hybridization, A Practical Approach, IRL Press, Washington, D.C. (1985), each of which is incorporated herein by reference. Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed. Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6, incorporated herein by reference. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. Those skilled in the art will recognize that hybridization solutions comprising salts other than sodium chloride and sodium citrate can also be used to obtain satisfactory hybridization conditions for practice of the methods of this invention. Various oligonucleotides suitable for capturing distinct polynucleotide sequences that are frequently found in transgenic plants are described in U.S. Patent Application Publication No. 20060127889.

III.c. Measurement

Once the polynucleotide of interest has been isolated, a variety of different measurement techniques can be employed to determine if the isolated polynucleotide is linked to a distinct polynucleotide. Both quantitative and qualitative measurement methods can be used to determine linkage. Hybridization-based nucleic acid detection techniques represent one measurement technique for determining linkage. These methods entail the specific hybridization of nucleic acid probes to the complementary sequence of the isolated polynucleotide and detection of hybridization. For certain types of linkage analyses, at least two nucleic acid hybridization probes are required. The first probe is capable of specifically binding to the isolated distinct polynucleotide whereas the second probe is capable of specifically binding to the distinct polynucleotide which may be linked to the isolated polynucleotide. These nucleic acid probes can be detectably labelled. Detectable labels, include but are not limited to, an enzyme, an isotope, a fluorophore, a lanthanide, a hapten, an oxidant, a reductant, a nucleotide and the like. Labeling of oligonucleotide probes with fluorescent labels can be accomplished as described in U.S. Pat. No. 6,838,244 or other references cited therein. When the nucleic acid probe is labelled with a hapten, it can be detected and quantitated by a coupling molecule that binds the hapten and permits detection. Coupling molecules that permit detection include, but are not limited to, antibodies, antibodies conjugated to enzymes, antibodies that are detectably labelled, antibodies labelled with fluorescent molecules, aptamers that recognize the hapten and other proteinaceous molecules that recognize the hapten. Haptens include, but are not limited to, biotin, digoxigenin, and the like that can be covalent linked to the nucleic acid probe. Proteinaceous molecules that recognize haptens include, but are not limited to, proteins such as streptavidin. In these hybridization-based assays, the amount of detectably labelled probe that is hybridized to the distinct polynucleotide is determined to provide a measurement of the amount of that distinct polynucleotide in the sample. Various oligonucleotides suitable for measuring distinct polynucleotide sequences that are frequently found in transgenic plants are described in U.S. Patent Application Publication No. 20060127889.

Measurement of distinct polynucleotide sequences can also be determined by quantitative reverse-transcriptase Polymerase Chain Reaction (qRT-PCR) techniques wherein the PCR product derived from the distinct polynucleotide can be detected. In general, such qRTPCR assays rely upon determination of a threshold cycle (or Cycle Threshold, referred to herein as a “CT” value), where the amount of the PCR product increases beyond a background level at a given number of PCR thermal cycles. Detection of the PCR product can be achieved by use of any of the aforementioned labelled polynucleotide hybridization probes, by use of an intercalating dye such as ethidium bromide or SYBR green, or use of a hybridization probe containing a fluorophore and a quencher such that emission from the fluorophore is only detected when the fluorophore is released by the 5′ nuclease activity of the polymerase used in the PCR reaction (i.e., a TaqMan™ reaction; Applied Biosystems, Foster City, Calif.) or when the fluorophore and quencher are displaced by polymerase mediated synthesis of the complementary strand (i.e., Scorpion™ or Molecular Beacon™ probes). Various methods for conducting qRT-PCR analysis to quantitate mRNA levels are well characterized and can be adapted for quantitation of distinct polynucleotides (Bustin, S. A.; Journal of Molecular Endocrinology 29, 23, 2002).

Fluorescent probes that are activated by the action of enzymes that recognize mismatched nucleic acid complexes (i.e., Invader™, Third Wave, Technologies, Madison, Wis.) can also be used to quantitate RNA. Those skilled in the art will also understand that nucleic acid quantitation techniques such as Quantitative Nucleic Acid Sequence Based Amplification (Q-NASBA™) can be used.

Various methods used to detect single nucleotide polymorphisms can also be used to determine if the isolated polynucleotide is linked to a distinct polynucleotide. For instance, distinct polynucleotides can be detected by hybridization to allele-specific oligonucleotide (ASO) probes as disclosed in U.S. Pat. Nos. 5,468,613 and 5,217,863. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe. Distinct polynucleotides can also be detected by probe ligation methods as disclosed in U.S. Pat. No. 5,800,944 where sequence of interest is amplified and hybridized to probes followed by ligation to detect a labeled part of the probe.

Microarrays can also be used for detection of polymorphisms and distinct polynucleotides, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening a plurality of polymorphisms. A single-feature polymorphism (SFP) is a polymorphism detected by a single probe in an oligonucleotide array, wherein a feature is a probe in the array. Typing of target sequences by microarray-based methods is disclosed in U.S. Pat. Nos. 6,799,122; 6,913,879; and 6,996,476.

Distinct polynucleotides can also be detected by probe linking methods as disclosed in U.S. Pat. No. 5,616,464 employing at least one pair of probes having sequences homologous to adjacent portions of the target nucleic acid sequence and having side chains which non-covalently bind to form a stem upon base pairing of said probes to said target nucleic acid sequence. At least one of the side chains has a photoactivatable group which can form a covalent cross-link with the other side chain member of the stem.

Other methods for detecting distinct polynucleotides include single base extension (SBE) methods. Examples of SBE methods include, but are not limited to, those disclosed in U.S. Pat. Nos. 6,004,744; 6,013,431; 5,595,890; 5,762,876; and 5,945,283. SBE methods are based on extension of a nucleotide primer that is immediately adjacent to a polymorphism to incorporate a detectable nucleotide residue upon extension of the primer. In certain embodiments, the SBE method uses three synthetic oligonucleotides. Two of the oligonucleotides serve as PCR primers and are complementary to sequence of the genomic DNA which flanks a region containing the polymorphism to be assayed. Following amplification of the region of the genome containing the polymorphism, the PCR product is mixed with the third oligonucleotide (called an extension primer) which is designed to hybridize to the amplified DNA immediately adjacent to the polymorphism in the presence of DNA polymerase and two differentially labeled dideoxynucleosidetriphosphates. If the polymorphism is present on the template, one of the labeled dideoxynucleosidetriphosphates can be added to the primer in a single base chain extension. The allele present is then inferred by determining which of the two differential labels was added to the extension primer. Homozygous samples will result in only one of the two labeled bases being incorporated and thus only one of the two labels will be detected. Heterozygous samples have both alleles present, and will thus direct incorporation of both labels (into different molecules of the extension primer) and thus both labels will be detected.

Distinct polynucleotides can also be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′→3′ exonuclease activity cleaves the probe and separates the reporter dye from the quencher dye resulting in increased fluorescence of the reporter.

III.d. Sequencing of Isolated Polynucleotides and Flanking Regions

Following separation and trapping of DNA sequences, amplification of the trapped DNA can produce sufficient quantities of the trapped DNA to enable that DNA to be sequenced. A particular gene sequence isolated by this method can also be sequenced. There are various reasons for sequencing a piece of genomic DNA isolated directly from a genome, including determining the site of a transgene insertion and resequencing specific genomic regions to look for single nucleotide polymorphisms.

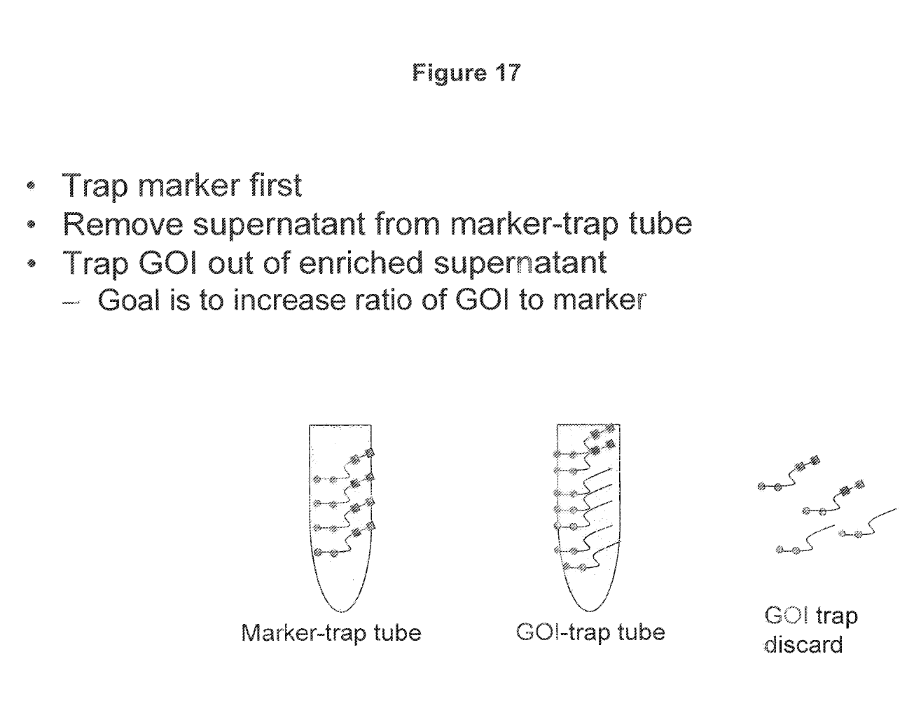

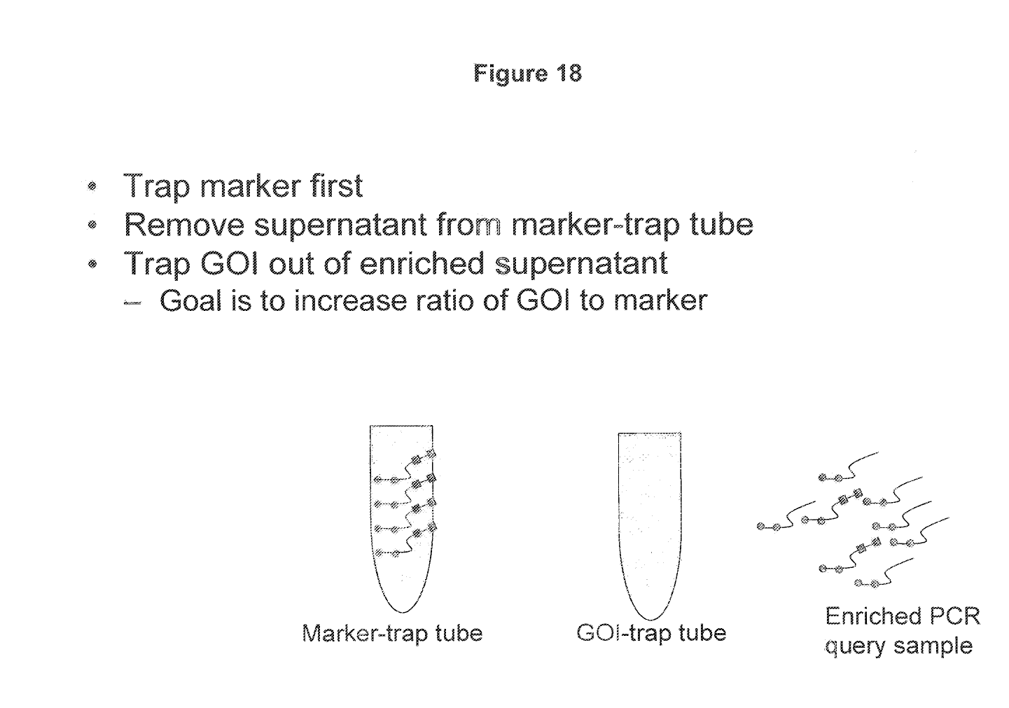

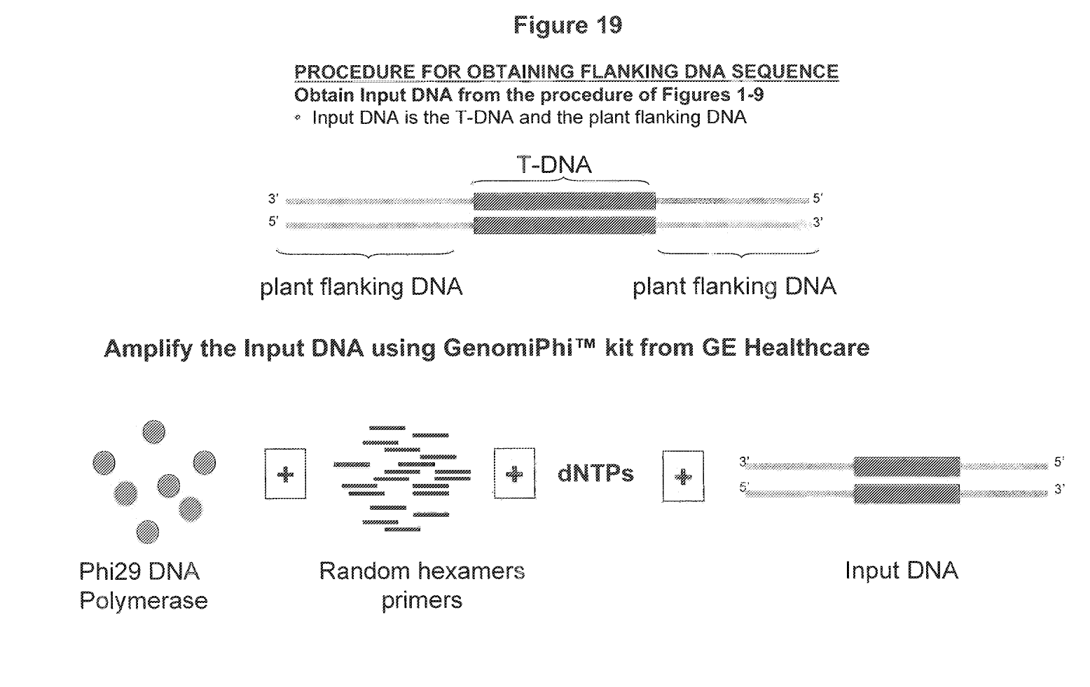

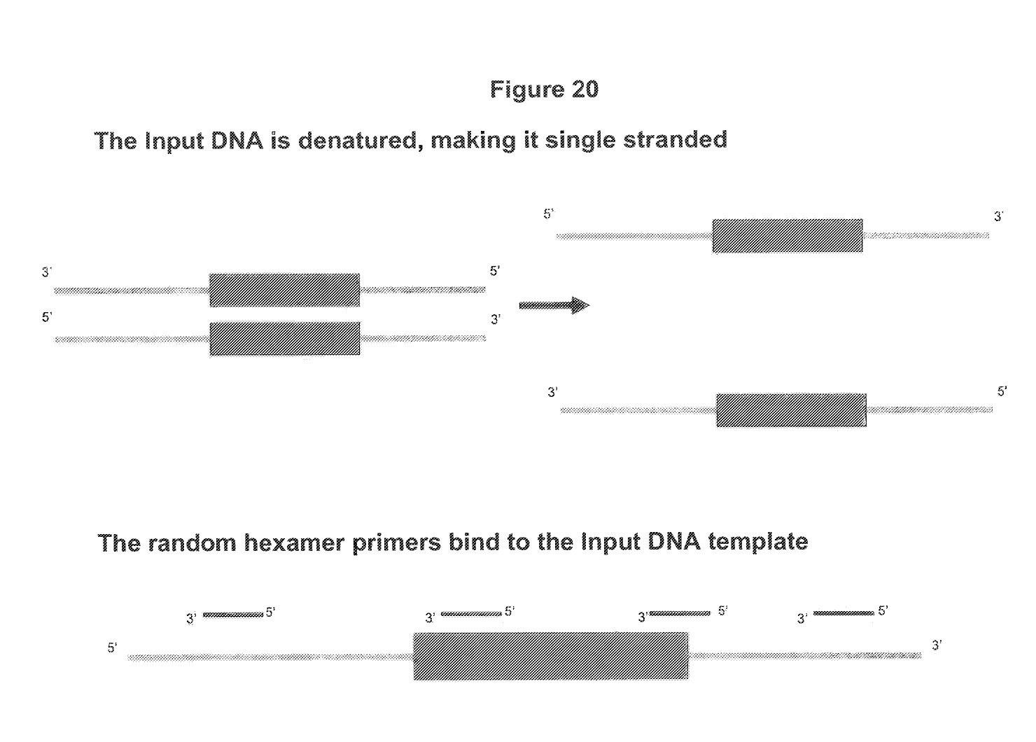

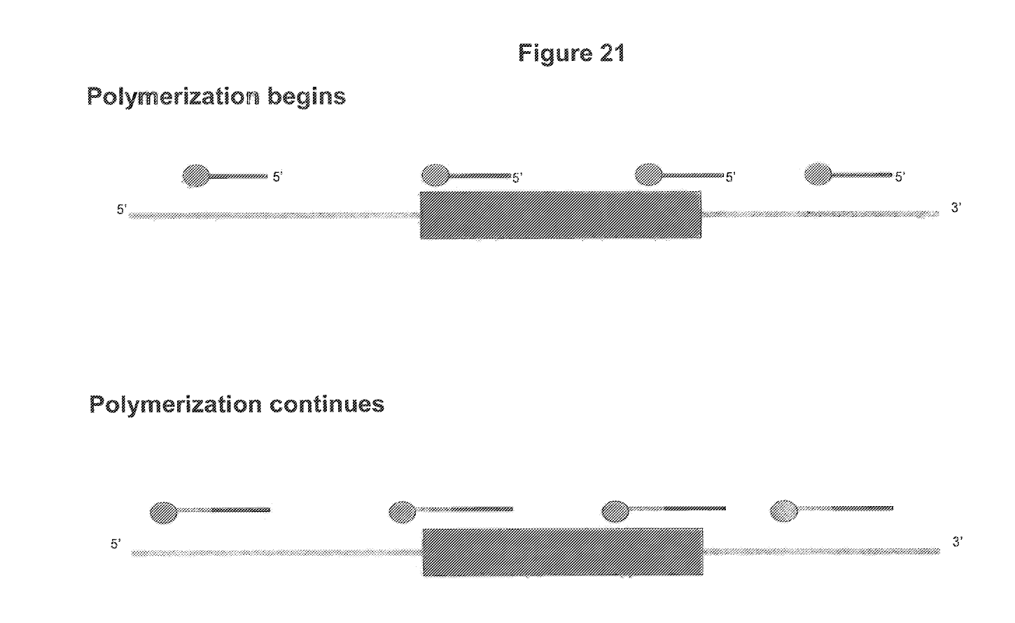

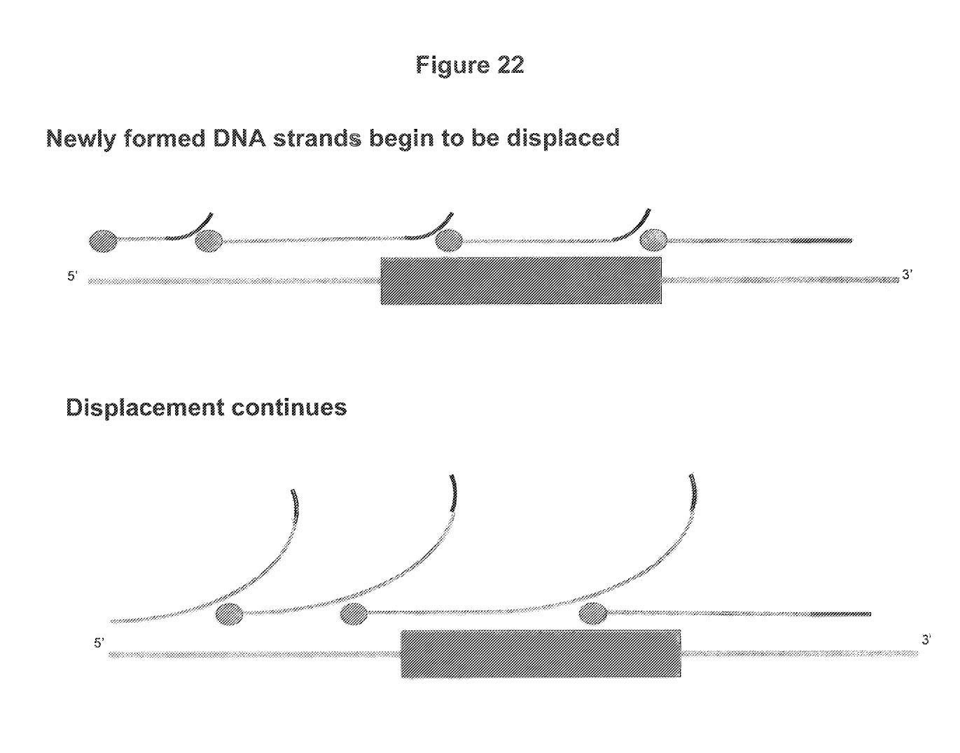

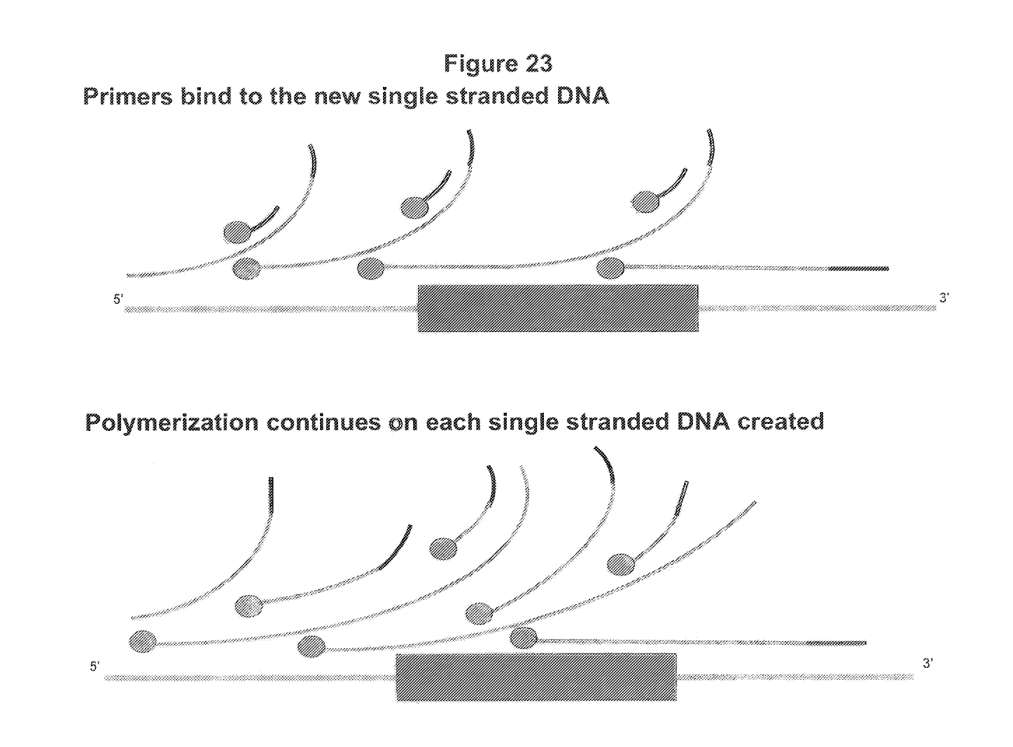

Amplification can be achieved using a sequence-independent DNA amplification technique (e.g. Phi 29 DNA polymerase) and random primers, a DNA fragment with limited known sequence (sufficient only for a trap to be made) could be amplified and completely sequenced. An exemplary illustration of sequence-independent DNA amplification is shown in FIGS. 17-22.

In the event that sequence data was desired, and random DNA amplification was not desirable (for reasons of specificity), the trapped DNA could be circularized by a DNA ligating enzyme (e.g. T4 DNA ligase), and a primer specific to a known element of the trapped DNA sequence (e.g. the gene of interest) could be used as a primer sequence to initiate amplification by any of a variety of DNA polymerases (e.g. Phi 29 DNA polymerase, Bst DNA polymerase, Taq polymerase). The use of the Phi 29 DNA polymerase in rolling circle amplification methods has been described (Dean et al., Genome Res. 2001 11: 1095-1099). Alternatively, one could also use a sequence specific primer in conjunction with a suitable DNA polymerase to obtain quantities of template sufficient for sequencing. Suitable DNA polymerases for use with sequence specific primers include but are not limited to, a Phi 29 DNA polymerase.

In the event that sequence was desired, enough DNA could be directly trapped to enable sequencing without resorting to any amplification technique. This could be done by trapping large concentrations of DNA in one vessel (e.g. microcentrifuge tube) or in multiple smaller vessels (e.g. 96-, 384-, or 1536-well microtiter plate).

One application of this sequencing method is flank sequence identification or isolation. Flank sequence analysis is the practice of determining the insertion location of a transgene. This is important in plant biotechnology for providing information to government regulatory agencies as flanking DNA sequence analysis must be done before a product can be marketed. Flank analysis is currently done using a variety of different methods, none of which is especially conducive to high throughput analysis. Furthermore, the process is laborious enough that the plants tend to be many generations post-transformation before flank analysis is complete. Since it costs a considerable amount of money to propagate and test plants in the field, and since plants are frequently discarded based on flank results, earlier flank sequence analysis is highly desirable.

III.e. Analysis

Once data are generated by a nucleic acid measurement technique using the methods of the invention, the data must be analyzed to determine which samples are linked and which are unlinked. It is contemplated that any technique that provides for the measurement of the relative amounts of the two distinct polynucleotides can be used to provide the data for analysis. Analysis of the data entails calculation of a relationship between the first and the second distinct polynucleotides to determine the linkage status of the two distinct polynucleotides. The calculated relationship comprises any comparison of the relative amounts of the two distinct polynucleotides. Relationships include, but are not limited to, ratios of the relative amounts of the two distinct polynucleotides, differences in the amounts of the two distinct polynucleotides, graphical representations of the relative amounts of the two distinct polynucleotides, or any other numerical representation of the relative amounts of the two distinct polynucleotide.

Data analysis can be done by a variety of ways to compare the results. In the case of PCR analyses, the data are captured and analyzed by CT (Cycle Threshold). Lower initial concentrations of the target DNA that is amplified will result in a higher CT value, indicating it took more cycles of PCR to amplify it sufficiently to be detectable. A lower CT value indicates that a higher concentration of the target DNA that is amplified was present at the initiation of the PCR reaction. Methods and compositions suitable for quantifying distinct polynucleotides that are frequently found in transgenic plants by quantitative PCR are described in U.S. Patent Application Publication No. 20060127889.

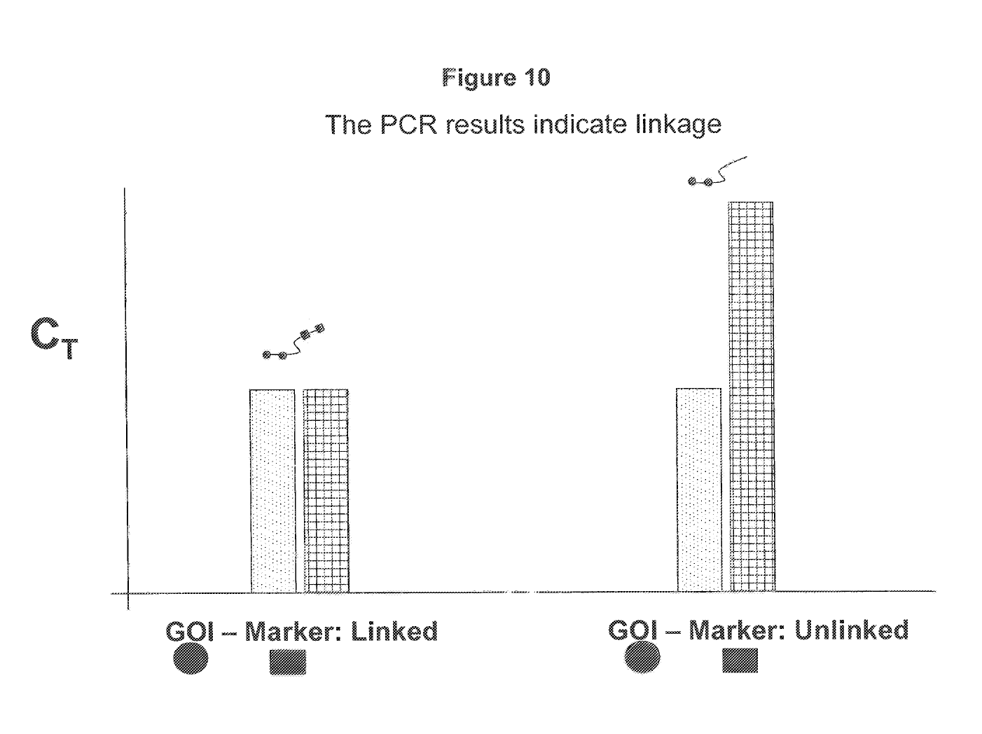

In one preferred embodiment, the PCR results from the trapped test sequence (first distinct polynucleotide) and the untrapped sequence (second distinct polynucleotide) are graphically displayed next to each other in a bar graph representation. An example of this type of representation is seen in FIG. 10 where the two distinct polynucleotides comprise a gene-of interest or GOI and a marker. If the two distinct polynucleotides are both present on the DNA fragment trapped by the method, then the CT (Cycle Threshold) for each are similar (i.e. the ratio of each is about 1 to 1), the data would indicate that the two distinct polynucleotides are linked (i.e. each trapped copy of the first distinct polynucleotide is associated with a copy of the second distinct polynucleotide). An unlinked DNA sample will have more of the first distinct polynucleotide present than the marker, since only the first distinct polynucleotide is trapped and retained; no second distinct polynucleotide copies are associated with a first distinct polynucleotide copies. Thus, the CT for the first distinct polynucleotide will be lower than the CT for the second distinct polynucleotide, as shown in FIG. 10. While there is some variation based on different efficiencies of the two PCR reactions, there is a significant difference between the linked and unlinked samples. Methods for obtaining matched sets of PCR primers and, when needed, detector oligonucleotides, with similar Tm values for their target sequences have been described. Such matched sets of PCR primers and/or detector oligonucleotides can be validated by running known amounts of the respective target templates (i.e. the two distinct polynucleotides) to obtain optimized primer sets that will yield CT values that reflect the relative amounts of input target templates.

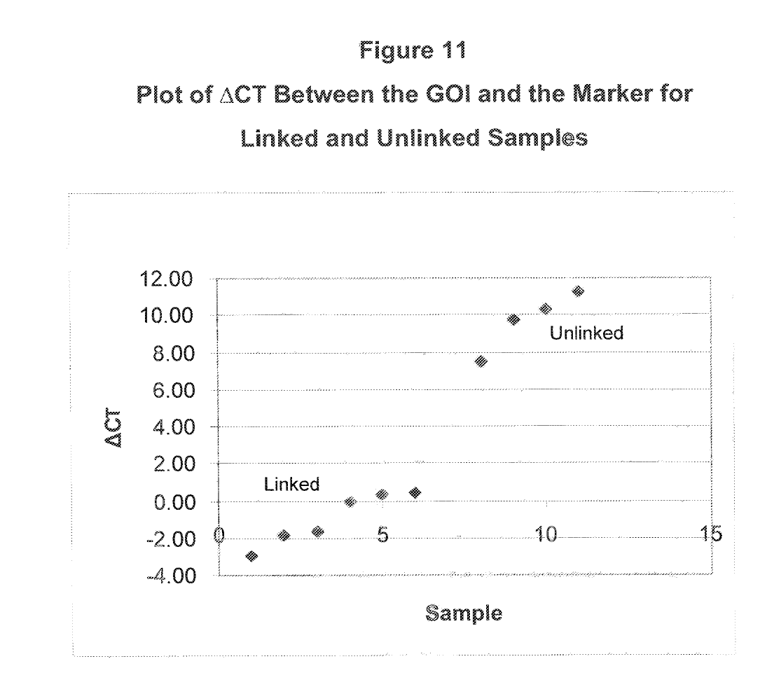

In another preferred embodiment, the difference in CT between the two distinct polynucleotides (for example, a GOI and the marker) can be expressed as ‘ΔCT’, in which the differences in CT indicate the linkage status. A higher ΔCT indicates an unlinked sample, while a lower ΔCT indicates a linked sample, as shown in FIG. 11.

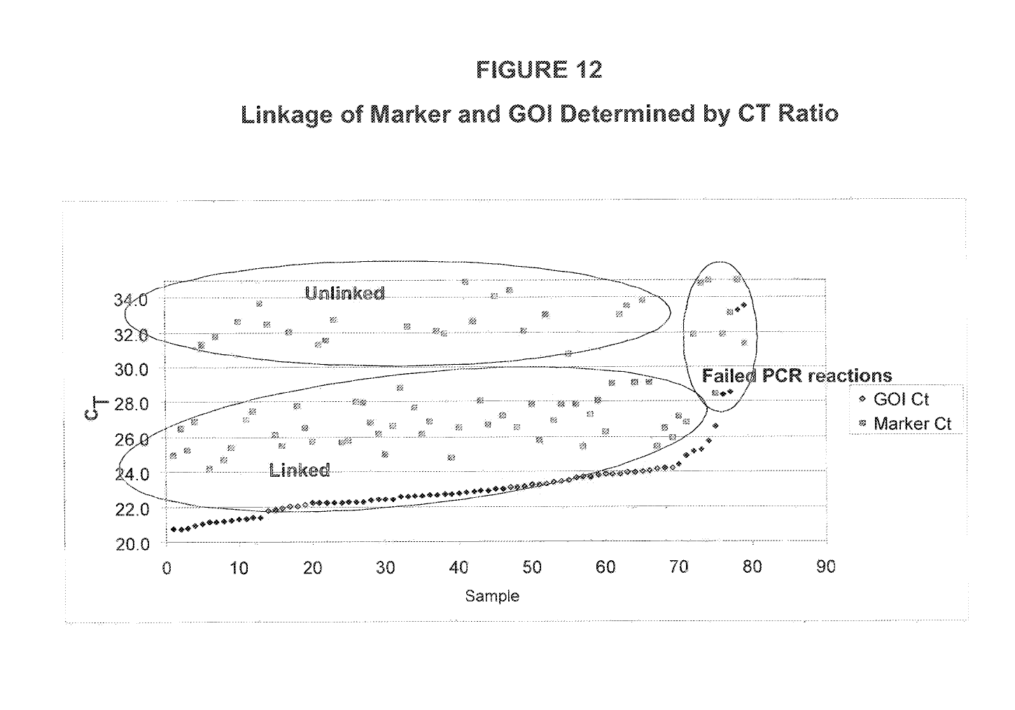

In yet another preferred embodiment, linked and unlinked polynucleotides can also be distinguished by comparing the CT of the two distinct polynucleotides (i.e. for example, by comparing a Marker CT with the GOI CT). Identification of unlinked polynucleotides is made by choosing those samples that have markedly higher CT's for the marker than for the GOI transgene; these are unlinked samples or events. Linked samples or events have CT's for the marker that are closer in value to those of the GOI. A certain number of samples may yield failed PCR reaction where any one or combination of the reactants used or samples provided do not support linkage analysis. Such failed reactions can be identified by a variety of criteria such as an increase in the CT value obtained for the first “trapped” distinct polynucleotide relative to range of typical CT values obtained for one or more positive control samples. Data from such failed reactions is typically discarded. An example of this type of scoring is shown in FIG. 12.

In still another preferred embodiment, analysis can be done numerically rather than graphically. Each of the methods described above can be accomplished by analyzing tabular data rather than graphs. In this case numerical values for the two distinct polynucleotides can be compared to determine if they are present in roughly equivalent amounts.

Those skilled in the art will recognize that the aforementioned analyses can be performed in parallel with various control samples (i.e. samples where linkage or flanking sequences are known). It is further contemplated that the numerical values that are compared to obtain an association or relationship, such as a ratio, between the distinct polynucleotides that are measured can be obtained by any method that provides for a reliable measurement of the relative amounts of the two distinct polynucleotides, and is not restricted or limited in any way to measurements obtained by PCR analyses or other techniques.

### IV. ALTERNATIVE METHODS FOR DETECTING LINKAGE

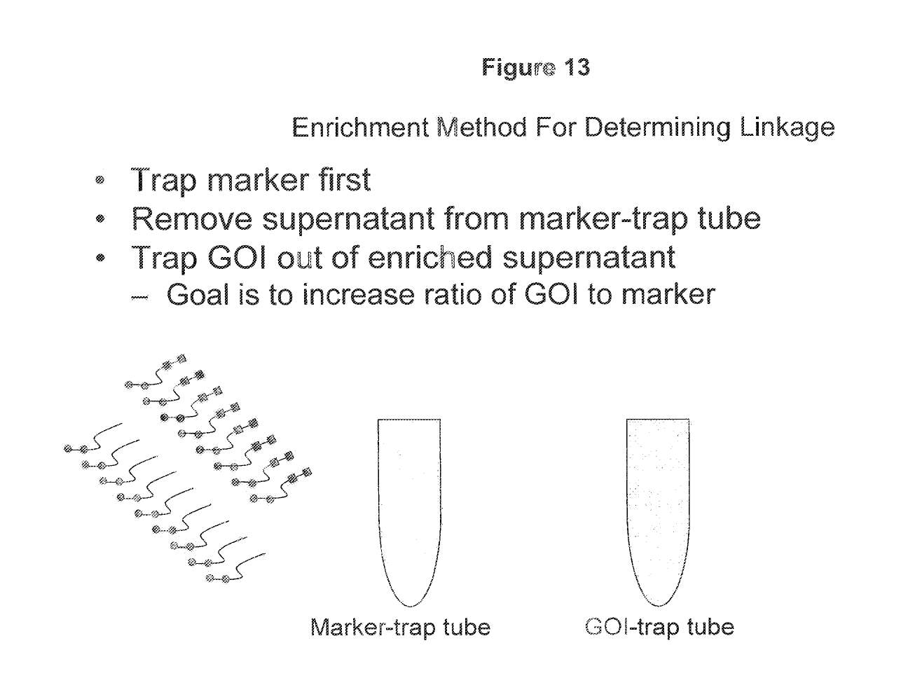

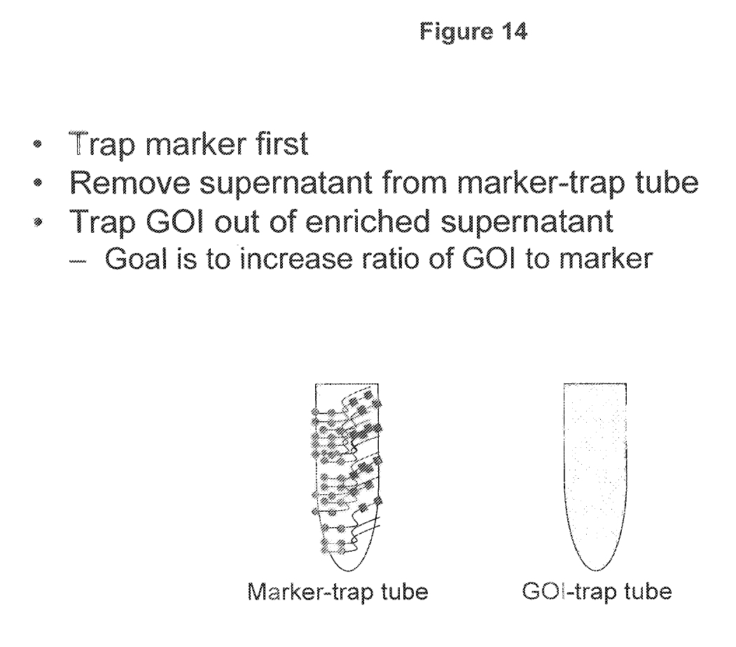

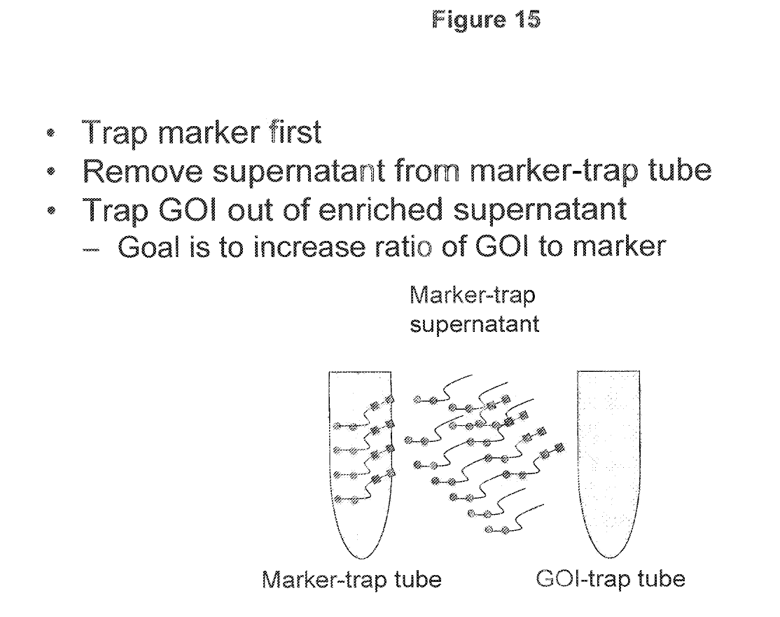

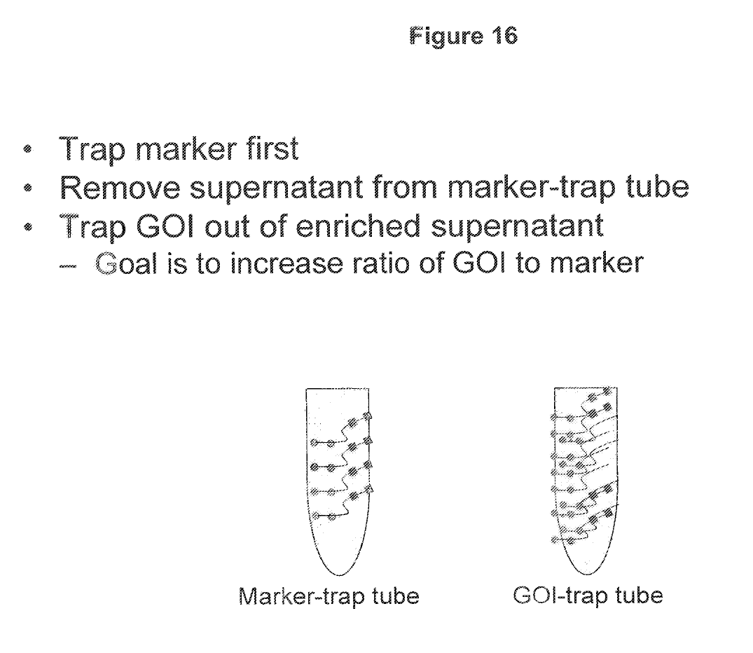

In other embodiments of the invention, methods where a distinct polynucleotide are enriched are used prior to isolating that distinct polynucleotide and determining if that polynucleotide is linked to a second distinct polynucleotide. One embodiment of this method is illustrated in FIGS. 13-18. This particular method is useful in obtaining linkage data when multiple copies of a distinct polynucleotide are present in a sample such that certain copies of that distinct polynucleotide are linked to a second distinct polynucleotide while other copies of that distinct polynucleotide in the sample are not linked to a second distinct polynucleotide. In practice, this situation can at least arise in instances where the distinct polynucleotide is a gene of interest that has inserted at two distinct locations in the genome of a host organism, such that one or more copies of that gene of interest are linked to the co-transformed marker gene at one location of the genome, but one or more other copies of the gene of interest located at another position in the genome are not linked to the marker. In such instances, capturing the first distinct polynucleotide and analyzing for linkage of the second polynucleotide would likely yield equivocal results that would not reveal the presence of the copy or copies of the distinct polynucleotide that are unlinked to a second distinct polynucleotide.

To address this issue that could arise in certain samples, an alternative method where the sample is first depleted for the second polynucleotide is proposed (see FIGS. 13-18). Depletion could be effected by capturing the second distinct polynucleotide with a suitable “capture hybridization probe”, thus depleting the sample of the second polynucleotide (and any copies of the first polynucleotide that are linked to the second polynucleotide). The Supernatant or solution containing and enriched for other copies of the first polynucleotide (which is unlinked) can then be subjected to standard methods of the invention (i.e. polynucleotide isolation, measurement and analysis) to determine linkage.

### V. APPLICATION OF THE METHODS OF THE INVENTION IN ANALYZING TRANSGENIC PLANTS

The methods of this invention can be applied to any of the commonly used methods of obtaining transgenic plants to rapidly identify plants where the plant expression cassette comprising the gene of interest is not linked to the plant expression cassette(s) comprising a selectable or scoreable marker. The methods of the invention can also be used to isolate or sequence the endogenous plant genomic DNA flanking the insertion site of the exogenous gene of interest in the transgenic plant. First, expression vectors suitable for expression of the gene of interest in various dicot and monocot plants are introduced into a plant, a plant cell or a plant tissue using transformation techniques as described herein. Genes of interest include but are both limited to, genes that provides an agronomic trait comprising herbicide tolerance, increased yield, insect control, fungal disease resistance, virus resistance, nematode resistance, bacterial disease resistance, mycoplasma disease resistance, modified oils production, high oil production, high protein production, germination and seedling growth control, enhanced animal and human nutrition, low raffinose, environmental stress tolerance, increased digestibility, industrial enzyme production, pharmaceutical peptides and small molecule production, improved processing traits, proteins improved flavor, nitrogen fixation, hybrid seed production, reduced allergenicity, biopolymers, or biofuel production. Next a transgenic plant containing or comprising the gene of interest expression cassette is obtained by regenerating that transgenic plant from the plant, plant cell or plant tissue that received the expression vector. This plant is then analyzed by the methods of this invention to determine if the gene of interest is linked to the selectable or scoreable marker gene that was co-introduced with the gene of interest. Transgenic plants expressing genes of interest contemplated herein include, but not limited to, barley, corn, oat, rice, rye, sorghum, turf grass, sugarcane, wheat, alfalfa, banana, broccoli, bean, cabbage, canola, carrot, cassava, cauliflower, celery, citrus, cotton, a cucurbit, eucalyptus, flax, garlic, grape, onion, lettuce, pea, peanut, pepper, potato, poplar, pine, sunflower, safflower, soybean, strawberry, sugar beet, sweet potato, tobacco, tomato, ornamental, shrub, nut, chickpea, pigeon pea, millets, hops, and pasture grass plants.

Plant transformation vectors typically comprise plant expression cassettes that provide for expression of genes of interest, selectable marker genes, and scoreable marker genes. The construction of expression cassettes for use in monocotyledonous plants or dicotyledonous plants is well established. Expression cassettes are DNA constructs where various promoter, coding, and polyadenylation sequences are operably linked. In general, expression cassettes typically comprise a promoter that is operably linked to a sequence of interest which is operably linked to a polyadenylation or terminator region. In certain instances including, but not limited to, the expression of transgenes in monocot plants, it may also be useful to include an intron sequence. When an intron sequence is included, it is typically placed in the 5′ untranslated leader region of the transgene. In certain instances, it may also be useful to incorporate specific 5′ untranslated sequences in a transgene to enhance transcript stability or to promote efficient translation of the transcript. Any of these aforementioned sequences can be used to devise appropriate hybridization probes for isolating or measuring a distinct polynucleotide.

The DNA constructs that comprise the plant expression cassettes described above are typically maintained in various vectors. Vectors contain sequences that provide for the replication of the vector and covalently linked sequences in a host cell. For example, bacterial vectors will contain origins of replication that permit replication of the vector in one or more bacterial hosts. Agrobacterium-mediated plant transformation vectors typically comprise sequences that permit replication in both E. coli and Agrobacterium as well as one or more “border” sequences positioned so as to permit integration of the expression cassette into the plant chromosome. Such Agrobacterium vectors can be adapted for use in either Agrobacterium tumefaciens or Agrobacterium rhizogenes. Selectable markers encoding genes that confer resistance to antibiotics are also typically included in the vectors to provide for their maintenance in bacterial hosts. The methods of this invention can also be used to determine if the bacterial selectable markers or any other extraneous sequences in the vector have been incorporated into the genome of the transgenic plant at the same genomic location as the gene of interest. Other extraneous sequences include but are not limited to, bacterial origins of replication, polylinker sequences, and/or plasmid vector backbone sequences that do not include the gene of interest. Although the commonly used methods of plant transformation typically include steps aimed at reducing the frequency with which the undesirable extraneous sequences have integrated into the plant genome, those steps occasionally fail. The methods describe herein can be used to determine if the extraneous sequences have integrated at the same genomic location as the genes of interest.

Plant expression cassettes comprising genes of interest, selectable markers and scoreable markers can be introduced into the chromosomes of a host plant via methods such as Agrobacterium-mediated transformation, Rhizobium-mediated transformation, Sinorhizobium-mediated transformation, particle-mediated transformation, DNA transfection, DNA electroporation, or “whiskers”-mediated transformation. Suitable methods for transformation of plants include any method by which DNA can be introduced into a cell, such as by electroporation as illustrated in U.S. Pat. No. 5,384,253; microprojectile bombardment as illustrated in U.S. Pat. Nos. 5,015,580; 5,550,318; 5,538,880; 6,160,208; 6,399,861; and 6,403,865; Agrobacterium-mediated transformation as illustrated in U.S. Pat. Nos. 5,635,055; 5,824,877; 5,591,616; 5,981,840; and 6,384,301; and protoplast transformation as illustrated in U.S. Pat. No. 5,508,184, etc. Aforementioned methods of introducing transgenes are well known to those skilled in the art and are described in U.S. Patent Application No. 20050289673 (Agrobacterium-mediated transformation of corn), U.S. Pat. No. 7,002,058 (Agrobacterium-mediated transformation of soybean), U.S. Pat. No. 6,365,807 (particle mediated transformation of rice), and U.S. Pat. No. 5,004,863 (Agrobacterium-mediated transformation of cotton). Through the application of techniques such as these, the cells of virtually any plant species may be stably transformed, and these cells developed into transgenic plants. Other techniques that may be particularly useful in the context of cotton transformation are disclosed in U.S. Pat. Nos. 5,846,797; 5,159,135; and 6,624,344; and techniques for transforming Brassica plants in particular are disclosed, for example, in U.S. Pat. No. 5,750,871; and techniques for transforming soybean are disclosed in for example in Zhang et al., 1999, and U.S. Pat. No. 6,384,301; and techniques for transforming corn are disclosed in WO9506722. Methods of using bacteria such as Rhizobium or Sinorhizobium to transform plants are described in Broothaerts, et al., Nature. 2005, 10; 433:629-33. It is further understood that the plant expression vector can comprise cis-acting site-specific recombination sites recognized by site-specific recombinases, including Cre, Flp, Gin, Pin, Sre, pinD, Int-B13, and R. Methods of integrating DNA molecules at specific locations in the genomes of transgenic plants through use of site-specific recombinases can then be used (U.S. Pat. No. 7,102,055). Those skilled in the art will further appreciate that any of these gene transfer techniques can be used to introduce the expression vector into the chromosome of a plant cell, a plant tissue or a plant.

The use of plant transformation vectors comprising two separate T-DNA molecules, one T-DNA containing the gene or genes of interest and another T-DNA containing a selectable and/or scoreable marker gene are also contemplated. In these two T-DNA vectors, the plant expression cassette or cassettes comprising the gene or genes of interest are contained within one set of T-DNA border sequences and the plant expression cassette or cassettes comprising the selectable and/or scoreable marker genes are contained within another set of T-DNA border sequences. In certain embodiments, the T-DNA border sequences flanking the plant expression cassettes comprise both a left and a right T-DNA border sequence that are operably oriented to provide for transfer and integration of the plant expression cassettes into the plant genome. In other embodiments, a tandem 2 T-DNA vector can be used to obtain transgenic plants with unlinked insertions of the gene of interest and the selectable or scoreable marker into the plant host chromosome as described in U.S. patent application Ser. No. 10/190,217. In the tandem 2 T-DNA vector, the gene of interest is contained within one set of Agrobacterium border sequences and the selectable marker and plasmid maintenance elements are located outside of the border sequences. When used with a suitable Agrobacterium host in Agrobacterium-mediated plant transformation, either the two T-DNA vector or tandem 2 T-DNA vector provides for integration of one T-DNA molecule containing the gene or genes of interest at one chromosomal location and integration of the other T-DNA containing the selectable and/or scoreable marker into another chromosomal location. Transgenic plants containing both the gene(s) of interest and the selectable and/or scoreable marker genes are first obtained by selection and/or scoring for the marker gene(s) and screened for expression of the genes of interest. Distinct lines of transgenic plants containing both the marker gene(s) and gene(s) of interest are subsequently out-crossed to obtain a population of progeny transgenic plants segregating for both the marker gene(s) and gene(s) of interest. Progeny plants containing only the gene(s) of interest can be identified by any combination of DNA, RNA or protein analysis techniques. Methods for using two T-DNA vectors have been described in U.S. Pat. Nos. 6,265,638; 5,731,179; and U.S. Patent Application Publication No. 2003110532A1, and U.S. Patent Application Publication No. 20050183170A1. Methods for using tandem T-DNA vectors have been described in U.S. patent application Ser. No. 10/190,217.

Transgenic plants are typically obtained by co-introduction of the gene of interest and a selectable gene into a plant cell, a plant tissue or a plant by any one of the methods described above, and regenerating or otherwise recovering the transgenic plant under conditions requiring expression of said selectable marker gene for plant growth. The selectable marker gene can be a gene encoding a neomycin phosphotransferase protein, a phosphinothricin acetyltransferase protein, a glyphosate resistant 5-enol-pyruvylshikimate-3-phosphate synthase (EPSPS) protein, a hygromycin phosphotransferase protein, a dihydropteroate synthase protein, a sulfonylurea insensitive acetolactate synthase protein, an atrazine insensitive Q protein, a nitrilase protein capable of degrading bromoxynil, a dehalogenase protein capable of degrading dalapon, a 2,4-dichlorophenoxyacetate monoxygenase protein, a methotrexate insensitive dihydrofolate reductase protein, and an aminoethylcysteine insensitive octopine synthase protein. The corresponding selective agents used in conjunction with each gene can be: neomycin (for neomycin phosphotransferase protein selection), phosphinotricin (for phosphinothricin acetyltransferase protein selection), glyphosate (for glyphosate resistant 5-enol-pyruvylshikimate-3-phosphate synthase (EPSPS) protein selection), hygromycin (for hygromycin phosphotransferase protein selection), sulfadiazine (for a dihydropteroate synthase protein selection), chlorsulfuron (for a sulfonylurea insensitive acetolactate synthase protein selection), atrazine (for an atrazine insensitive Q protein selection), bromoxinyl (for a nitrilase protein selection), dalapon (for a dehalogenase protein selection), 2,4-dichlorophenoxyacetic acid (for a 2,4-dichlorophenoxyacetate monoxygenase protein selection), methotrexate (for a methotrexate insensitive dihydrofolate reductase protein selection), or aminoethylcysteine (for an aminoethylcysteine insensitive octopine synthase protein selection).

Transgenic plants can also be obtained by co-introduction of a gene of interest and a scoreable marker gene into a plant cell by any one of the methods described above, and regenerating the transgenic plants from transformed plant cells that test positive for expression of the scoreable marker gene. Scoreable marker genes are any genes that provide for simple destructive or non-destructive expression assays. The scoreable marker gene can be a gene encoding a beta-glucuronidase protein, a green fluorescent protein, a yellow fluorescent protein, a red fluorescent protein, a beta-galactosidase protein, a luciferase protein derived from a luc gene, a luciferase protein derived from a lux gene, a sialidase protein, streptomycin phosphotransferase protein, a nopaline synthase protein, an octopine synthase protein or a chloramphenicol acetyl transferase protein.

When the expression vector is introduced into a plant cell or plant tissue, the transformed cells or tissues are typically regenerated into whole plants by culturing these cells or tissues under conditions that promote the formation of a whole plant (i.e., the process of regenerating leaves, stems, roots, and, in certain plants, reproductive tissues). The development or regeneration of transgenic plants from either single plant protoplasts or various explants is well known in the art (Horsch, R. B. et al., 1985). This regeneration and growth process typically includes the steps of selection of transformed cells and culturing selected cells under conditions that will yield rooted plantlets. This initial regenerated plant or plantlet are referred to as an “R₀” plant, while subsequent generations of plants derived from that “R₀” plant are referred to as “R₁”, “R₂”, or “Rₓ” plants, where “x” is the generation number of the plant relative to the initial regenerated parent. The resulting transgenic rooted shoots are thereafter planted in an appropriate plant growth medium such as soil. Alternatively, transgenes can also be introduced into isolated plant shoot meristems and plants regenerated without going through callus stage tissue culture (U.S. Pat. No. 7,002,058). When the transgene is introduced directly into a plant, or more specifically into the meristematic tissue of a plant, seed can be harvested from the plant and selected or scored for presence of the transgene. In the case of transgenic plant species that reproduce sexually, seeds can be collected from plants that have been “selfed” (self-pollinated) or out-crossed (i.e., used as a pollen donor or recipient) to establish and maintain the transgenic plant line. Transgenic plants that do not sexually reproduce can be vegetatively propagated to establish and maintain the transgenic plant line. As used here, transgenic plant line refers to transgenic plants derived from a transformation event where the transgene has inserted into one or more locations in the plant genome. In a related aspect, the methods of the present invention can also be applied to a seed produced by the transformed plant, a progeny from such seed, and a seed produced by the progeny of the original transgenic plant, produced in accordance with the above process. Such progeny and seeds will have an gene of interest stably incorporated into their genome, and such progeny plants will inherit the traits afforded by the introduction of a stable transgene in Mendelian fashion.

The methods of the instant application can be applied to any transgenic plant of any generation. However, it is particularly advantageous to apply the methods of the invention to “R₀” plants or plantlets as the information provided can be used to cull undesirable transgenic events (i.e. those where the gene of interest and the selectable or scoreable marker are linked) from a population. The sample used in the methods of this invention can be obtained from any portion of the transgenic plant including, but not limited to, the leaf, root, flower, stem, or any combination thereof.

In the event of multiple gene insertions in the same plant (e.g. plant genome), the flanking sequence could be used to establish which plants had transgenes in particular loci (i.e. positions in the plant genome). If an R1 plant has 4 copies of a gene, many of the 2-copy plants are useful, but only if both copies of the gene are at the same locus. To establish this, additional Southern-Blot analyses, called locus Southerns, are typically done. This is expensive and time consuming. As flanking sequences must be established for provision of data to regulatory agencies that govern commercialization of transgenic plants, the high-throughput technique for establishing flank sequences at the R1 stage described herein enables both flanking DNA regions and loci to be determined simultaneously.

### VI. KITS

The present invention contemplates kits for determining linkage of distinct polynucleotides in samples that use the methods of the invention. In certain particular embodiments contemplated herein, the methods and kits detect linkage of a gene of interest to a commonly used selectable or scoreable marker. A kit may contain one reagent that provides for capture of a first distinct polynucleotide and instructions for the use the reagent in determining linkage. The provided reagent(s) can be radio-, spectrophotometrically-, fluorescently- or enzymatically-labeled. The provided reagents can also be labelled with a suitable hapten. The provided reagents may include a substrate that is converted to a product that can be detected by spectrophotometry, luminometry, or fluorescence. The kit can contain a hybridization probe that can be used to capture a distinct polynucleotide. The kit can also contain a detectably labelled probe for measuring a distinct polynucleotide.

The reagent(s) of the kit may be provided as a liquid solution, attached to a solid support or as a dried powder. Preferably, when the reagent(s) are provided in a liquid solution, the liquid solution is an aqueous solution. Preferably, when the reagent(s) provided are attached to a solid support, the solid support can be a bead, chromatographic media, a test plate having a plurality of wells (i.e. a microtiter plate), an array, or a slide. Alternatively, the reagents can be in a format that provides for attachment to the solid support. For example, an oligonucleotide reagent can be labelled with a hapten such as biotin that provides for attachment to a solid support that is coupled to avidin. When the reagent(s) provided are a dry powder, the powder can be reconstituted by the addition of a suitable solvent, that may be provided.

The container will generally include a vial into which the capture or detection reagent may be placed. The reagent is preferably suitably aliquotted. The kits of the present invention will also typically include a means for containing the reagent containers in close confinement for commercial sale. Such containers may include injection or blow-molded plastic containers into which the desired vials are retained.

### EXAMPLES

The following disclosed embodiments are merely representative of the invention, which may be embodied in various forms. Thus, specific structural and functional details disclosed herein are not to be interpreted as limiting.

### Example 1

### Sample Preparation and Separation of Distinct Polynucleotide Sequences

This example describes the preparation of a sample and the separation of the distinct polynucleotide sequences of interest from a plant sample.

Genomic DNA was extracted using a filter Dellaporta DNA extraction process (Dellaporta, S. L., et al., 1983. A plant DNA minipreparation: version II. Pl. Molec. Biol. Reporter 1: 19-21). In other instances, standard phenol-chloroform extraction has also been used successfully. Briefly, lyophilized leaf tissue was placed in a 96-well sample box (Nunc, Inc.), 2 steel ball bearings were added to each well, the box was sealed with a cap map (Nunc, Inc., Rochester, N.Y., US) and the box was shaken for approximately 3 minutes on a Harbil paint shaker to pulverize the tissue. Extraction buffer (1% final concentration in 400 microliters extraction buffer (American Bioanalytical, Inc., Natick, Mass., US; Catalogue No. CU14139-20000) was added to each well, and the mixture was shaken again for approximately 1 minute and then incubated for approximately 45 minutes at 65° C. To each well, 255 microliters of isopropanol and 135 microliters of % M potassium acetate were added and the mixture was again shaken for approximately 1 minute. The box was centrifuged (Jouan, Inc, Winchester, Va., USA, Model KR-422) for 15 minutes at approximately 4000×g, and the supernatant was drained and discarded. The precipitated DNA pellets were allowed to dry, and then 200 microliters of room temperature 70% ethanol was added. The plate was shaken vigorously on an orbital shaker (Lab-Line Instruments, Melrose Pk, Ill., US model 4625) for approximately 30 seconds, and then spun again at approximately 4000×g for 10 minutes. The ethanol was drained, the pellet was allowed to dry and resuspended in 50-200 microliters of water (tris-EDTA may also be used).

Between 1 and 4 μg of DNA was digested with a restriction enzyme that did not cut within the DNA construct to be tested, but that digested the DNA to average fragment sizes of between 2 and 20 kilobases (e.g. BglII for soy DNA) and incubated overnight at 37° C. A PCR annealing reaction was set up by combining 1 μg of digested DNA, 2 nMol biotinylated traps oligonucleotides, 1×PCR buffer and sterile water added to bring the final volume to 20 microliters per well of the microtiter plate. The plate was sealed and briefly spun in a centrifuge. The annealing reaction was run on a PCR thermocycler, using the conditions of 95° C. for 15 minutes, slowly ramping down to 62° C., holding for 5 minutes, then holding at 15° C.

After annealing, streptavidin-magnetizable beads (New England Biolabs, Beverly, Mass., USA, catalog #S1420S) were added to each well. Beads were washed and then resuspended in wash/binding buffer (0.5 M NaCl, 20 mM Tris-HCl pH 7.5, 1 mM EDTA) prior to addition to the DNA-trap reactions. The reaction was incubated at 37° C. for one hour on the thermal cycler.

The beads were then magnetized, washed and the beads resuspended. The reaction plate was placed on a magnet for 2 minutes to allow all beads to magnetize (i.e. separate the beads from the supernatant, and the supernatant was removed from each well while the microtiter plate was on the magnet. The plate was removed from the magnet and 25 microliters of wash/binding buffer as added to each well. Each well was then manually pipetted up and down 10 times to break up the bead pellet, using fresh pipet tips for each column of samples. The plate was then incubated at room temperature for 3 minutes. This entire step was then repeated one time.

The final step involved magnetizing and eluting the trapped DNA. The plate was magnetized for 2 minutes, and the supernatant removed while the plate was on the magnet. About 20 uL water was added to each well, and each well resuspended. The plate was sealed and incubated for 95° C. for 5 minutes on a thermal cycler. The plate was then removed and magnetized for 2 minutes. Eluted DNA was transferred to a new plate and used immediately for subsequent analysis by TaqMan™ Realtime PCR (Applied Biosystems, Foster City, Calif., US).

Attempts were made to dilute the magnetic beads. It was found that a concentration of 25% of the manufacturer's recommendation was not significantly different from the recommended concentration, but lower concentrations produced less difference in the CT between marker and GOI.

Attempts were made to vary the concentration of the traps (i.e. biotin-labelled capture oligonucleotides complementary to the GOI). It was found that approximately an order of magnitude concentration in either direction still allowed the technique to work, but it was possible to reduce assay effectiveness by adding too much or too little trap.

We attempted to vary the time of trapping and annealing; we found that the times currently used were the minimal times possible to allow accurate results.

### Example 2

### Measurement of Distinct Polynucleotides

TaqMan™ assays were run by transferring 2 microliters of the eluted DNA to each of three different wells of either a 96-well or 384-well Realtime assay plate (Applied Biosystems (ABI) 96 or 384 well reaction plates (cat #4309849). 8 microliters of PCR mastermix (ABI 2X Universal Master Mix (cat #4304437), containing primers and probe at the appropriate concentration for each validated reaction) was added to each well. An optically-clear cover (ABgene cat #Ab0558) was placed over the plate, and the reaction was cycled (Applied Biosystems 7900HT) according to the following parameters: 50° C. for 2 minutes, 95° C. for 10 minutes, and then 35 cycles of 95° C. for 15 seconds and 60° C. for 1 minute. Three separate PCR reactions were done for each sample of DNA: the reference gene of interest (GOI) reaction, and reactions for the selectable marker and the construct backbone. Data were read as Realtime cycle threshold (CT) by the thermocycler.

### Example 3

### Analysis of Measurement Data

Upon completion of the PCR reactions, the data were downloaded into the Microsoft Excel™ software program (Microsoft, Inc, Redmond, Wash., US), and arranged to provide comparisons of the CT values. A graphical representation of the data obtained in this experiment are shown in FIG. 12. Linked and unlinked events were distinguished by comparing the Marker CT with the GOI CT. Event selection was made by choosing those events that had markedly higher CT's than those of the marker; these were unlinked events. Linked events had CT's closer to those of the GOI. A certain population failed PCR reactions; these may or may not be kept, depending on the number of plants needed.

### Example 4

### Sample Preparation, Separation, and of Sequencing of Distinct Polynucleotides

This example describes the preparation of a sample and the separation of the distinct polynucleotide sequences of interest from a plant sample for sequencing.

Genomic DNA was extracted using a filter Dellaporta DNA extraction process (Dellaporta, S. L., et al., 1983. A plant DNA minipreparation: version II. Pl. Molec. Biol. Reporter 1: 19-21). In other instances, standard phenol-chloroform extraction has also been used successfully. Briefly, lyophilized leaf tissue was placed in a 96-well sample box (Nunc, Inc.), 2 steel ball bearings were added to each well, the box was sealed with a cap map (Nunc, Inc., Rochester, N.Y., US) and the box was shaken for approximately 3 minutes on a Harbil paint shaker to pulverize the tissue. Extraction buffer (1% final concentration in 400 microliters extraction buffer (American Bioanalytical, Inc., Natick, Mass., US; Catalogue No. CU14139-20000) was added to each well, and the mixture was shaken again for approximately 1 minute and then incubated for approximately 45 minutes at 65° C. To each well, 255 microliters of isopropanol and 135 microliters of % M potassium acetate were added and the mixture was again shaken for approximately 1 minute. The box was centrifuged (Jouan, Inc, Winchester, Va., USA, Model KR-422) for 15 minutes at approximately 4000×g, and the supernatant was drained and discarded. The precipitated DNA pellets were allowed to dry, and then 200 microliters of room temperature 70% ethanol was added. The plate was shaken vigorously on an orbital shaker (Lab-Line Instruments, Melrose Pk, Ill., US model 4625) for approximately 30 seconds, and then spun again at approximately 4000×g for 10 minutes. The ethanol was drained, the pellet was allowed to dry and resuspended in 50-200 microliters of water (tris-EDTA may also be used).

To achieve the appropriate amount of DNA for sequencing (approximately 100 to 1000 nanograms of post-trapping genomic DNA mass), one of two techniques was used, isothermal amplification or bulk trapping. For the amplification process, between 1 and 4 μg of DNA was digested with a restriction enzyme that produces fragments of the length to be sequenced (hundreds of bases up to megabases), and does not cut within the desired elements to be sequenced (e.g. BglII for soy DNA) and incubated overnight at 37° C. A PCR annealing reaction was set up by combining 1 ug of digested DNA, 2 nMol biotinylated traps oligonucleotides, 1×PCR buffer and sterile water added to bring the final volume to 20 microliters per well of the microtiter plate. The plate was sealed and briefly spun in a centrifuge. The annealing reaction was run on a PCR thermocycler, using the conditions of 95° C. for 15 minutes, slowly ramping down to 62° C., holding for 5 minutes, then holding at 15° C.

After annealing, streptavidin-magnetizable beads (New England Biolabs, Beverly, Mass., USA, catalog #S1420S) were added to each well. Beads were washed and then resuspended in wash/binding buffer (0.5 M NaCl, 20 mM Tris-HCl pH 7.5, 1 mM EDTA) prior to addition to the DNA-trap reactions. The reaction was incubated at 37° C. for one hour on the thermal cycler.

The beads were then magnetized, washed and the beads resuspended. The reaction plate was placed on a magnet for 2 minutes to allow all beads to magnetize (i.e. separate the beads from the supernatant, and the supernatant was removed from each well while the microtiter plate was on the magnet. The plate was removed from the magnet and 25 microliters of wash/binding buffer as added to each well. Each well was then manually pipetted up and down 10 times to break up the bead pellet, using fresh pipet tips for each column of samples. The plate was then incubated at room temperature for 3 minutes. This entire step was then repeated one time.

The final step involved magnetizing and eluting the trapped DNA. The plate was magnetized for 2 minutes, and the supernatant removed while the plate was on the magnet. About 20 uL water was added to each well, and each well resuspended. The plate was sealed and incubated for 95° C. for 5 minutes on a thermal cycler. The plate was then removed and magnetized for 2 minutes. Eluted DNA was then used as the template for an isothermal amplification reaction, using General Electric's GenomiPhi kit (GE Healthcare Bio-Sciences Corp., Piscataway, N.J. USA). Briefly, approximately 20 ng of trapped and eluted DNA was added to the kit, and the reaction was run for 1 hour, per direction. The resulting amplified DNA was used as the template for a standard Sanger sequencing reaction.

For the bulk trapping process, approximately 1 mg of DNA was digested with a restriction enzyme that produces fragments of the length to be sequenced (hundreds of bases up to megabases), and does not cut within the desired elements to e sequenced (e.g. BglII for soy DNA) and incubated overnight at 37° C. A PCR annealing reaction was set up by combining 1 ug of digested DNA, 2 nMol biotinylated traps oligonucleotides, 1×PCR buffer and sterile water added to bring the final volume to 20 microliters per well of the microtiter plate. The plate was sealed and briefly spun in a centrifuge. The annealing reaction was run on a PCR thermocycler, using the conditions of 95° C. for 15 minutes, slowly ramping down to 62° C., holding for 5 minutes, then holding at 15° C.

After annealing, streptavidin-magnetizable beads (New England Biolabs, Beverly, Mass., USA, catalog #S1420S) were added to each well. Beads were washed and then resuspended in wash/binding buffer (0.5 M NaCl, 20 mM Tris-HCl pH 7.5, 1 mM EDTA) prior to addition to the DNA-trap reactions. The reaction was incubated at 37° C. for one hour on the thermal cycler.

The beads were then magnetized, washed and the beads resuspended. The reaction plate was placed on a magnet for 2 minutes to allow all beads to magnetize (i.e. separate the beads from the supernatant, and the supernatant was removed from each well while the microtiter plate was on the magnet. The plate was removed from the magnet and 25 microliters of wash/binding buffer as added to each well. Each well was then manually pipetted up and down 10 times to break up the bead pellet, using fresh pipet tips for each column of samples. The plate was then incubated at room temperature for 3 minutes. This entire step was then repeated one time.

The final step involved magnetizing and eluting the trapped DNA. The plate was magnetized for 2 minutes, and the supernatant removed while the plate was on the magnet. About 20 uL water was added to each well, and each well resuspended. The plate was sealed and incubated for 95° C. for 5 minutes on a thermal cycler. The plate was then removed and magnetized for 2 minutes. The resulting eluted DNA was used as the template for a standard Sanger sequencing reaction.

### Example 5

### High Throughput Enrichment and Sequencing of Long Target Nucleic Acid Fragments

Materials and Methods

Plant Growth

Arabidopsis thaliana (ecotype Columbia) plants were grown in an environmentally controlled growth chamber for a 16 hour day, 25° C./19° C. (day/night) and 70% relative humidity, where an irradiance of 50-120 W m-2 was provided by 1000 W lamps.

Binary Agrobacterium Vector Construction

An artificial miRNA gene that was designed to suppress the expression of GLABROUS1 (GL 1), a myb gene homolog required for the initiation of trichome development, was created and placed under the control of the CaMV 35S promoter in the pMON100616, a binary Agrobacterium construct, which also carries the CP4 EPSPS (5-enolpyruvylshikimate-3-phosphate synthase) as a selectable marker for transformed plants.

Plant Transformation

The T-DNA from the pMON100616 vector was introduced into Arabidopsis plants by Agrobacterium-mediated transformation (Clough and Bent, Plant J. 16(6):735-43). The T0 seeds were germinated in the soil and two-week old seedlings were sprayed with Roundup™ (41% active ingredient of glyphosate; Monsanto Co., St. Louis, Mo.). Seeds were collected from the resistant plants and homozygous plants were selected from the progeny plants.

Design of Traps

Traps (i.e. biotinylated oligonucleotides) were designed to be homologous to a portion of the target sequence and to have melting temperatures (Tm) of 65° C. or higher. Traps were 20 to 35 nucleotides in length and the GC content of traps was about 50%. Each trap was biotinylated on the 5′ end to allow later capture of target DNA and ordered from Invitrogen (Carlsbad, Calif., USA). The traps used in this study were CP4nno_AT_F252 (5′-biotin-CGGAGGATTGCTCGCTCCCGA-3′; SEQ ID NO:4) and CP4nno_AT_R1122 (5′-biotin-TTCGTCGCAGTCCACGCCGTT-3′; SEQ ID NO:5) for the transgene and flanking DNA regions. G1988 650 F (5′-biotin-GCTTTTGCGAGCTTTGTGGTGC-3′; SEQ ID NO:6) and G1988 1309 R (5′-biotin-CGTTTTCAGCCCATCCTTCCTCC-3′; SEQ ID NO:7) were used as traps for the for the native At3g21150 gene.

Preparation of Streptavidin-Coated Magnetic Beads

The high affinity binding that occurs between streptavidin and biotin allowed use of streptavidin-coated magnetic beads to capture of the hybridized DNA/biotin labeled probe complex. Prior to use, MagnaSphere™ Paramagnetic Particles (Promega, Madison, Wis., USA) were treated with a blocking step to decrease the nonspecific binding of DNA directly to the beads. The beads were washed three times with 6×SSC. Each wash entailed resuspension of the beads in 6×SSC followed by the use of a magnetic stand (Promega) to draw the beads aside, thus allowing for removal and disposal of the wash buffer. The beads were resuspended in a bead block buffer [0.2% I-Block™ (Applied Biosystems, Foster City, Calif., USA), 0.5% SDS in PBS (0.058 M Na₂HPO₄, 0.017 M NaH₂PO₄×H₂O, 0.068 M NaCl). The blocking solution and beads were gently mixed for 40-60 minutes at room temperature on a rocker platform. Three washes with 6×SSC followed the bead block and the blocked beads were then resuspended in 6×SSC.

Genomic DNA Extraction

Genomic DNA was isolated from leaf tissue of wild type or R6 plants following a modified CTAB method (Murray and Thompson, Nucleic Acids Res. 8(19):4321-5, 1980). Freeze-dried leaf tissue was ground with one 3 mm steel bead in a 15-ml Falcon tube. The samples were incubated at 65° C. in CTAB buffer containing 0.2% 2-mercaptoethanol and then extracted with Chl/IAA (25:1). After centrifugation at 5,700 rpm for 10 minutes, the aqueous phase was mixed with cold isopropanol to precipitate DNA. The DNA was recovered by centrifugation at 5,700 rpm (6100 g) for 10 minutes. The pellet was then washed in 70% Ethanol and re-suspended in TE (Tris/EDTA) buffer.

Restriction Digestion

Two micrograms of genomic DNA was digested with Sal I for the transgene, Xba I for native At3g21150 gene, or other suitable restriction endonucleases (10,000 U/mL, New England Biolabs (NEB), Ipswich, Mass., USA), at the appropriate temperature for at least 1 hour. After incubation, the digestion reaction was heated for 20 minutes at 65° C. to denature the enzymes if necessary.

Purification of Target Fragments

After digestion with a restriction endonuclease, the resulting DNA fragments were denatured at 95° C. for 15 minutes and then hybridized with the biotin-labeled traps at the hybridization temperature specific to the probe, typically 5-10° C. below Tm for 5 minutes in 1×PCR buffer (20 mM Tris HCl (pH 8.4) and 50 mM KCl). The pretreated streptavidin-coated magnetic beads were added directly to the hybridization reaction and incubated at 37° C. for 30 minutes to 3 hours to capture the hybridized target fragments. The beads and bound target fragments were washed at room temperature twice in the wash buffer (500 mM NaCl, 20 mM Tris-HCl and 1 mM EDTA). The target fragments were then eluted in TE buffer from the beads by incubating at 95° C. for 5 minutes.

Amplification of Target Fragments

The quality of the purified target fragments was assessed by duplexed real-time PCR on the target and an endogenous reference gene. The delta Ct (Ct reference-Ct target) normally was larger than 10. The purified target fragments were denatured at 95° C. for 3 minutes and then amplified at 30° C. for using random hexamer primers and the Phi29 DNA polymerase (GenomiPhi™; GE Healthcare, Piscataway, N.J.).

Fragmentation of Target Fragments

The target fragments of 9 kb and 15 kb were successfully amplified using the methods described in this Example. Three to five micrograms of the amplified products were chopped into small fragments by applying 30 psi of nitrogen for 2 minutes and 30 seconds in the Nebulization Buffer™ (Roche, Indianapolis, Ind., USA) using a nebulizer that was set in a wet ice-isopropanol bath. The nebulized DNA was purified using the MinElute™ PCR Purification Kit (Qiagen, Valencia, Calif., USA) and the small fragments were removed using AMPure™ SPRI beads (Agencourt Bioscience Corporation, Beverly, Mass., USA), following the suppliers' recommendations. The fragmented DNA samples were assessed for quality on a BioAnalyzer DNA 1000 LabChip (Agilent Technologies, Santa Clara, Calif.). The mean size should be between 400 and 800 bp with less than 10% being below 300 bp.

Construction of Shotgun Libraries

The fragmented DNA was end polished, added the adapters with multiplexing IDs and captured on the Library Immobilization Beads in the Library Binding Buffer (Roche). The beads were washed twice in the Library Wash Buffer (Roche) and then fill-in reaction was conducted on the beads at 37° C. for 20 minutes. After the reaction, the beads were washed twice in the Library Wash Buffer and the captured DNA was denatured in the Melt Solution (0.125 N NaOH) and the single-stranded template DNA (sstDNA) was purified using the MinElute™ PCR Purification Kit (Qiagen) and resolved in TE buffer. The single-stranded template DNA (sstDNA) was assessed for quality and quantity using a BioAnalyzer™ RNA 600 LabChip according to the manual (Agilent Technologies).

Sequencing of Libraries

The sstDNA molecules were captured and amplified on the beads using a GS emPCR™ Kit (Roche). After PCR, the beads carrying amplicons were collected using the Enrichment Beads™ (Roche). The amplicons on the beads were then annealed with the sequencing primer and sequenced on a Genome Sequencer™ FLX according to the manual (Roche).

Sequence Data Processing

The reads were separated according to the barcode sequences before the adaptor sequence was removed and submitted into gsMapper™ (Roche) for assembly and mapping.

Results

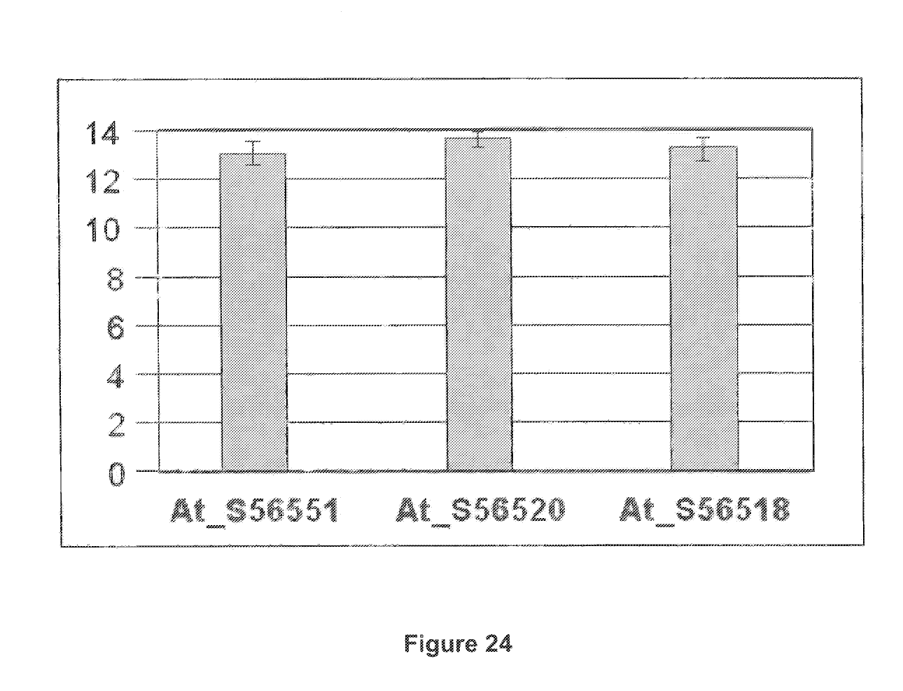

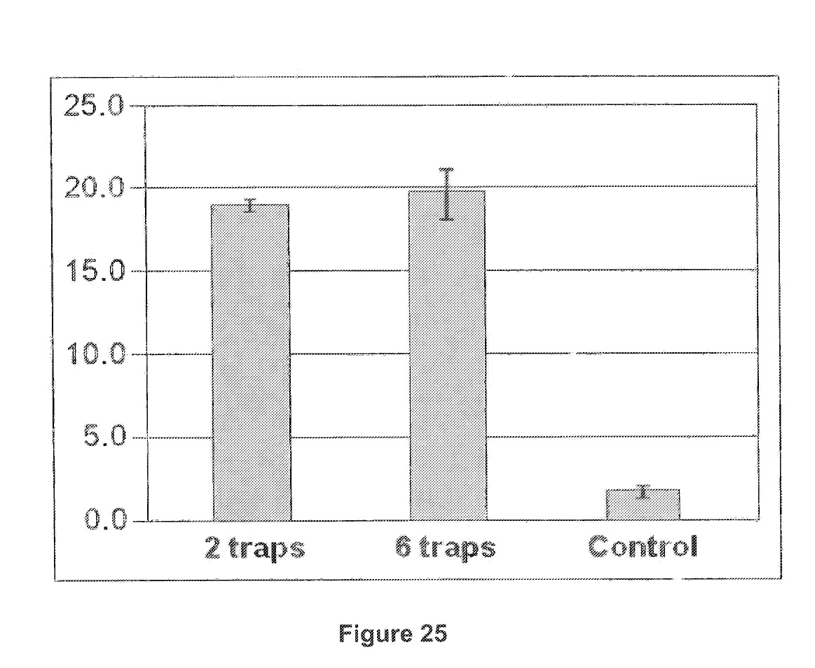

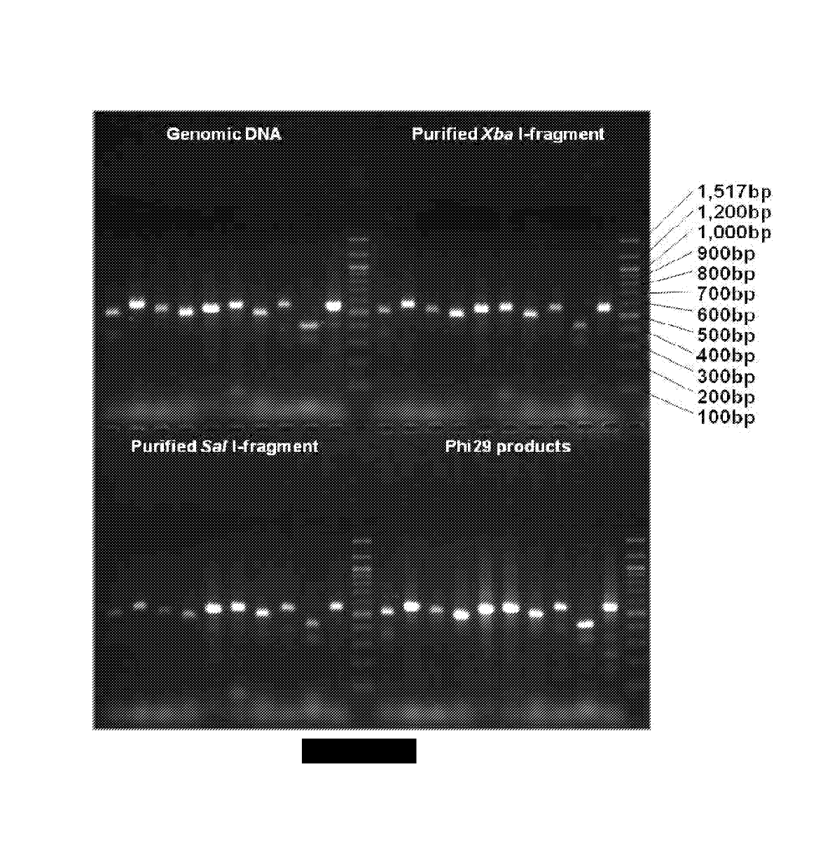

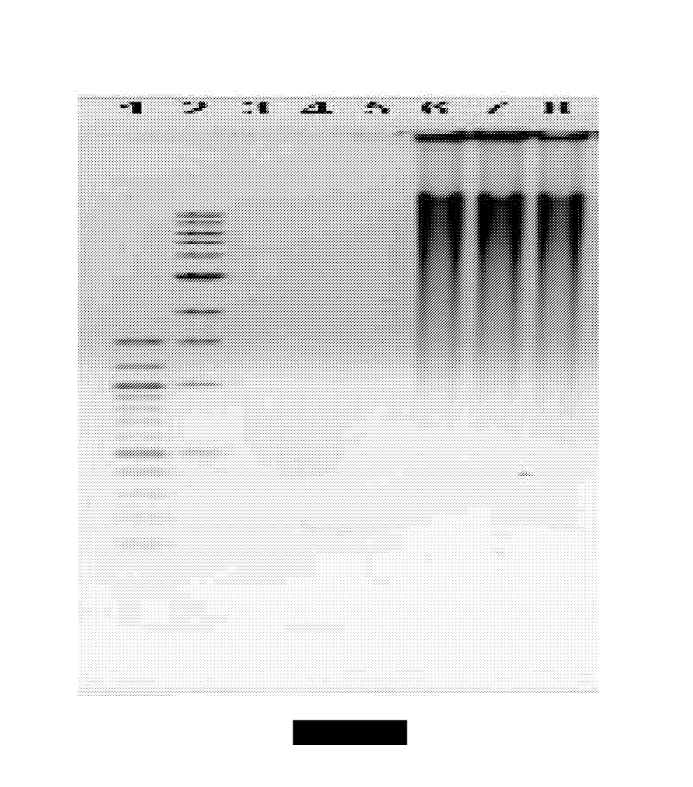

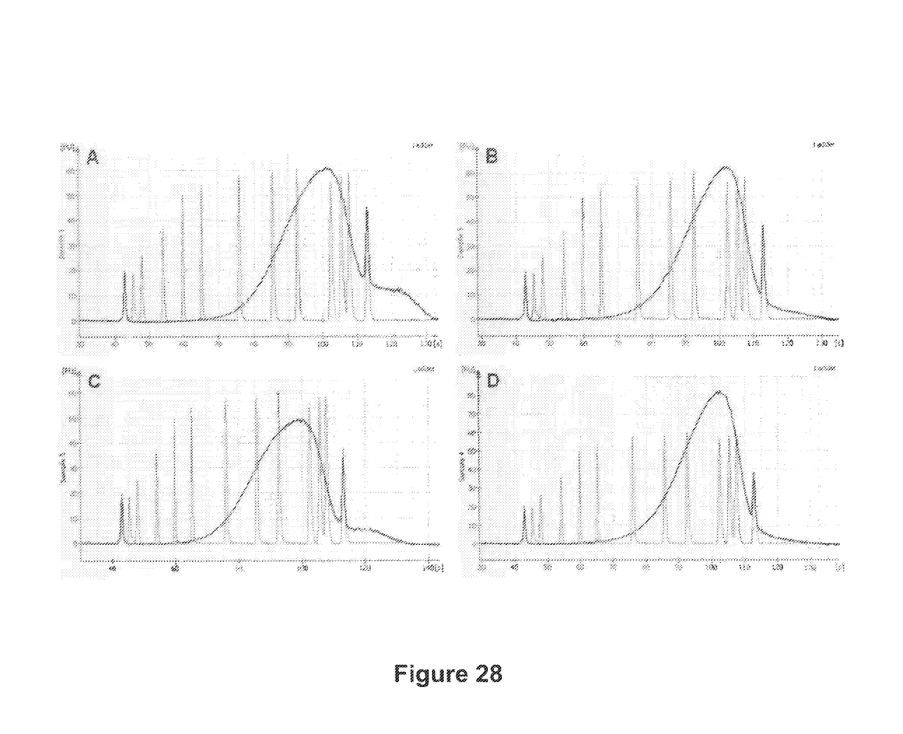

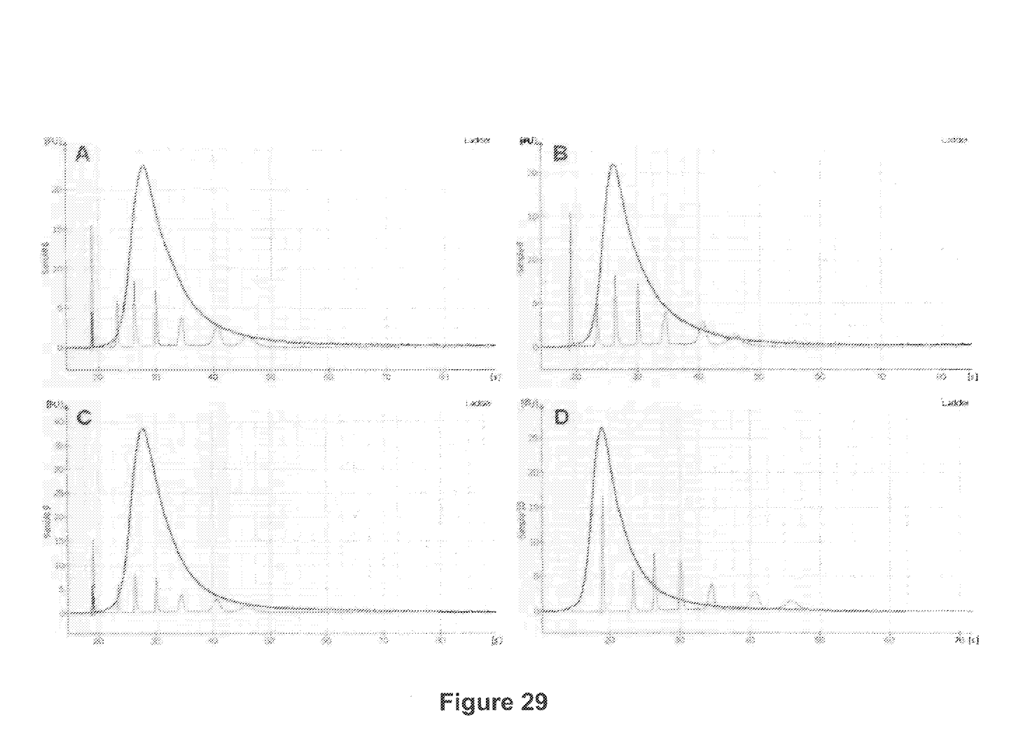

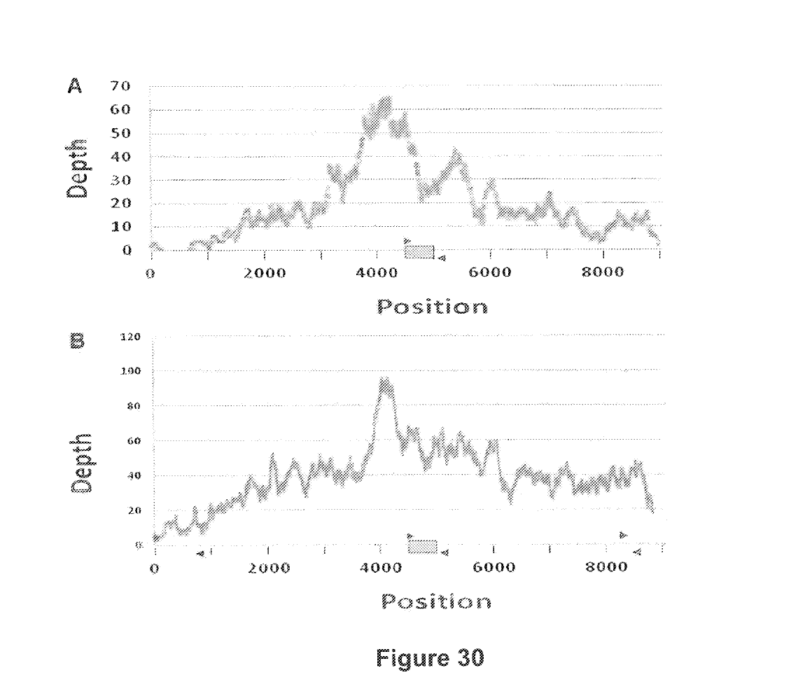

Real-time PCR analysis of the enriched genomic DNA captured with the trap oligonucleotides indicated more than 1000-times enrichment of the target fragments from 3 Arabidopsis transgenic events relative to a control non-target genomic sequence (reference sequence) after purification (FIG. 24). Pretreatment of streptavidin magnetic beads with 0.2% I-Block (a highly purified casein-based blocking reagent) and 0.5% SDS in PBS buffer could considerably prevent nonspecific binding of DNA and therefore reduce background relative to untreated beads (shown as Control in FIG. 25). Using 6 traps instead of 2 could increase enrichment efficiency. PCR analysis of the genomic DNA captured with the trap oligonucleotides indicated that the enriched DNA fragments were intact (FIG. 26). DNA electrophoresis results indicated that the enriched fragments of 9 kb and 15 kb were successfully amplified with the (FIG. 27). Electropherograms of the fragmented target DNA fragments generated by the nebulizer indicated that the majority was about 850 bp in length (FIG. 28). Electropherograms of the sstDNA indicated that the majority of that DNA was about 850 bp in length (FIG. 29). Sequence read distributions on the 8.9 kb target fragment carrying At3g21150 that were generated by sequencing of captured sstDNA fragments further indicated that a broader distribution of reads were obtained by using 6 trap oligonucleotides distributed across the 8.9 kB target fragment (FIG. 30, Panel B) relative to the distribution of reads obtained with just two trap oligonucleotides located in or near the gene of interest in the center of the fragment (FIG. 30, Panel A).

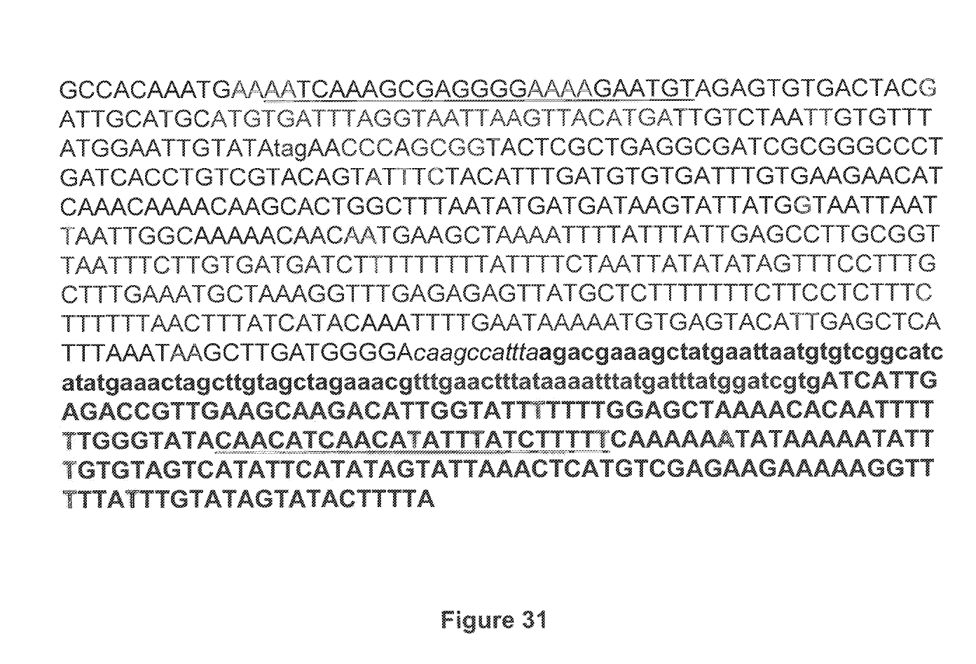

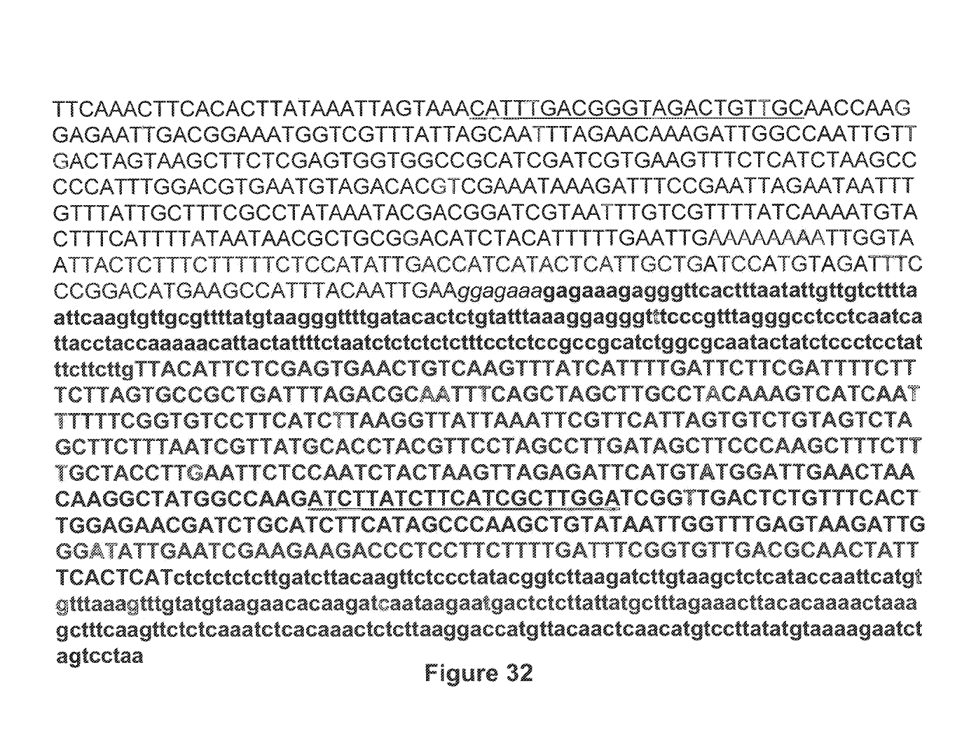

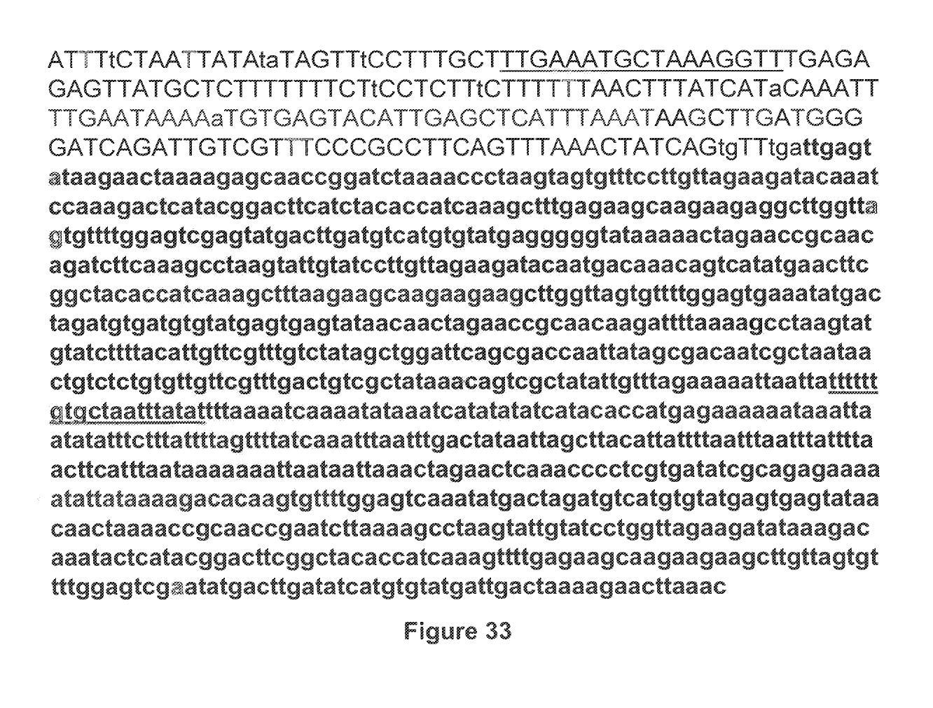

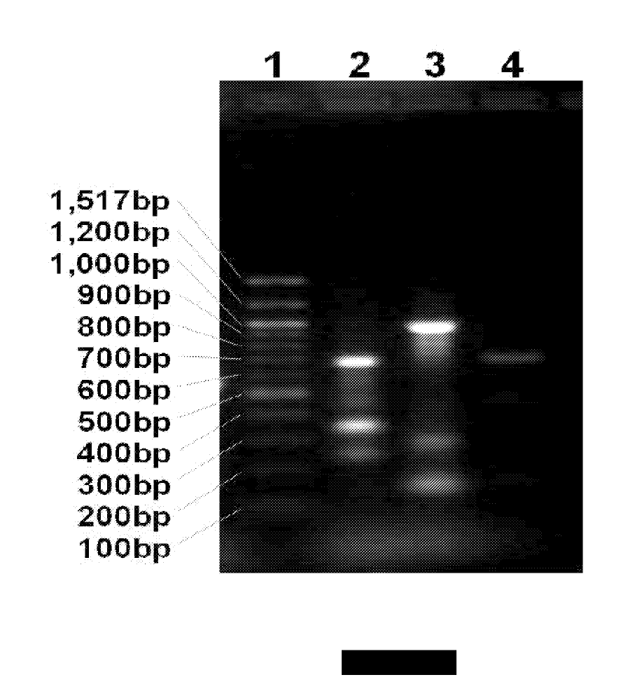

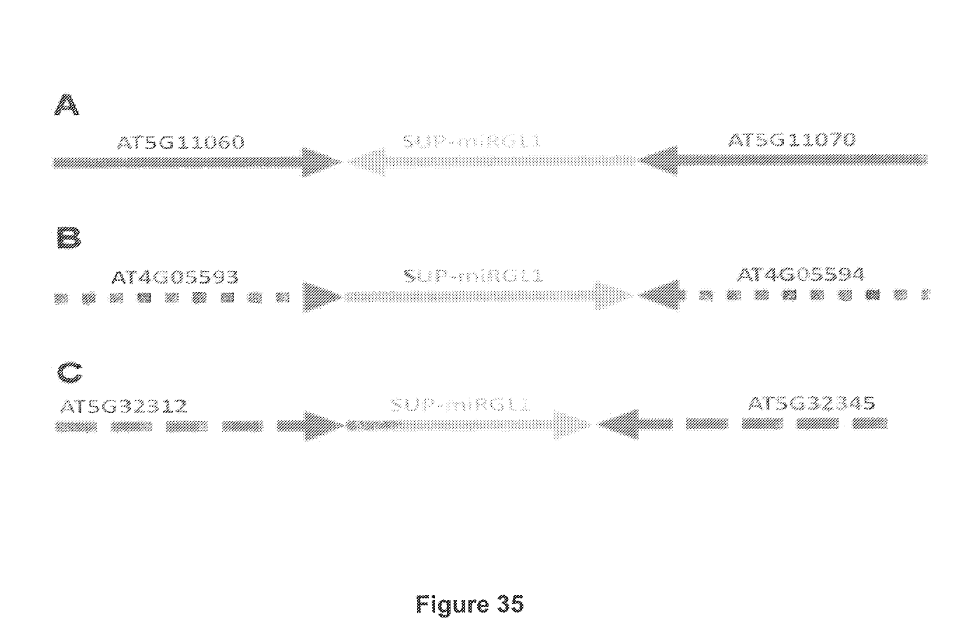

The sequences obtained from analysis of the Arabidopsis genomic DNA/T-DNA junction sequences of the three transgenic Arabidopsis events At_S56518, At_S56520, and At_S56551 are shown in FIGS. 31 (SEQ ID NO:1), 32 (SEQ ID NO:2), and 33 (SEQ ID NO:3), respectively. Examination of these sequences revealed that Arabidopsis events At_S56518 had inserted in a genomic region located between two typical Arabidopsis genes (AT5G11060 and AT5G11070). In contrast, the T-DNA insertion (SUP-miRGL1) in event At-S56520 was in a TY3/gypsy-like retrotransposon (AT4G05593 and AT4G05594). In the At-S56551 insertion event, the right border of the T-DNA insertion (SUP-miRGL1) was truncated and T-DNA insertion was adjacent to a TY3/gypsy-like retrotransposon (AT5G32345).

The insertion of At-S56520 and At-S56551 either within or adjacent to TY3/gypsy-like retrotransposon sequences is interesting in that certain plants harboring these transgene insertions display loss of the phenotype that is conferred by the active transgene. More specifically, the GLABROUS1 miRNA encoding transgene, when active, confers a glabrous phenotype (i.e. loss of trichomes on the leaves) similar to that observed in Arabidopsis plants that are homozygous for recessive mutations in the GLABROUS1 locus. The At-_S56520 transgene insertion event produced many off-type plants that lost the silenced phenotype that is indicative of an active transgene starting in generation 3 and continuing thru generation 6. The At-S56551 event has also produced some plants that appear to have lost the silenced phenotype conferred by the GLABROUS1 miRNA encoding transgene, but not as many as event ZM_S56520. In contrast, the At_S56518 event, which had inserted in a region located between two typical Arabidopsis genes and that is characterized by the absence of retrotransposon sequences, has displayed the silenced phenotype in six generations of progeny plants.

The insertion of a transgene either within or adjacent to retrotransposon sequences can thus be used to predict the potential stability of a transgene-conferred phenotype in successive generations of progeny transgenic plants, where plants comprising an insertion within or adjacent to a retrotransposon are predicted to exhibit decreased stability of transgene expression relative to plants comprising transgene insertions into regions comprising typical plant genes or comprising transgene insertions into regions that lack retrotransposon sequences.

From the examples given, the present invention thus provides methods and techniques useful for separating and analyzing polynucleotide sequences and methods useful in determining characteristics of transgenic plants. In particular, the present invention includes and provides high-throughput methods for analysis of transgene linkage in transformed plants, and kits for the same.

All patent and non-patent documents cited in this specification are incorporated herein by reference in their entireties, to the same extent as if each individual was specifically and individually indicated to be incorporated by reference. Documents cited herein as being available from the World Wide Web at certain internet addresses are also incorporated herein by reference in their entireties. Certain biological sequences referenced herein by their “NCBI Accession Number” can be accessed through the National Center of Biotechnology Information on the world wide web at ncbi.nlm.nih.gov.

As various modifications could be made in the methods herein described and illustrated without departing from the scope of the invention, it is intended that all matter contained in the foregoing description or shown in the accompanying drawings shall be interpreted as illustrative rather than limiting. Having illustrated and described the principles of the present invention, it should be apparent to persons skilled in the art that the invention can be modified in arrangement and detail without departing from such principles. All such modifications in arrangement and detail are considered to fall within the spirit and scope of the appended claims. Thus, the breadth and scope of the present invention should not be limited by any of the above-described exemplary embodiments, but should be defined only in accordance with the following claims appended hereto and their equivalents.

1.-67. (canceled)

68. A method for determining linkage of at least two distinct polynucleotides, comprising the steps of:
a. obtaining a sample comprising at least two distinct polynucleotides;
b. hybridizing at least one probe to a first distinct polynucleotide in said sample from (a) to obtain a hybridized polynucleotide complex comprising a first distinct polynucleotide;
c. separating said hybridized polynucleotide complex from said sample;
d. determining a measurable feature of said first distinct polynucleotide in the hybridized polynucleotide complex obtained in step (c) to a measurable feature in a second distinct polynucleotide;
e. comparing said measurable features from step (d); and
f. calculating a relationship between said first and said second distinct polynucleotides, wherein the results of said relationship are used to determine the linkage status of the two distinct polynucleotides.

69. The method of claim 68, wherein any of said polynucleotides are obtained from a transgenic plant selected from the group consisting of barley, corn, oat, sorghum, turf grass, sugarcane, wheat, alfalfa, banana, broccoli, bean, cabbage, canola, carrot, cassava, cauliflower, celery, citrus, cotton, a cucurbit, eucalyptus, flax, garlic, grape, onion, lettuce, pea, peanut, pepper, potato, poplar, pine, rye, rice, sunflower, safflower, soybean, strawberry, sugar beet, sweet potato, tobacco, tomato, ornamental, shrub, nut, millet, and pasture grass.

70. The method of claim 68, wherein said first or said second distinct polynucleotide is a transgenic polynucleotide of agronomic interest or is a polynucleotide that encodes or is operably linked to a selectable or scoreable marker gene.

71. The method of claim 68, wherein any of said probes is an oligonucleotide or an oligonucleotide coupled to biotin.

72. The method of claim 68, wherein at least one of said probes is immobilized on a solid support selected from the group consisting of a bead, a filter, a column, an array and a microtiter well.

73. The method of claim 72, wherein said bead is of a type selected from the group consisting of: magnetized, dye labelled, linked to a hapten, linked to a ligand, and combinations thereof.

74. The method of claim 68, wherein said separation is effected by a technique selected from the group consisting of a magnetic separation, bead sorting, electrophoretic separation, and buffer exchange, or any combination thereof.

75. The method of claim 68, wherein said measurable feature of said first or second distinct polynucleotide is selected from the group consisting of a Cycle Threshold (CT) value, a molecular weight of a defined sequence of said polynucleotides, a fluorescence value, a sample mass, a molarity and a polynucleotide sequence.

76. The method of claim 68, wherein the calculations provide a ratio of said measurable features, wherein a ratio of about 1 part of said first distinct polynucleotide sequence to about 1 part of said second distinct polynucleotide sequence in said separated isolated sample indicates that the two polynucleotide samples are linked, and wherein a ratio of about 1 part of said first distinct polynucleotide sequence to less than about 1 part of said second distinct polynucleotide sequence in said isolated sample indicates that the two polynucleotide samples are unlinked.

77. The method of claim 68, wherein said calculations provide a difference of said measurable features, wherein a higher value for the difference indicates that the first and second distinct polynucleotide samples are unlinked, and wherein a lower value for the difference indicates that the first and second distinct polynucleotide samples are linked.

78. The method of claim 68, wherein said measurable feature is obtained by a method selected from the group consisting of a symmetric polymerase chain reaction (PCR) assay, an asymmetric polymerase chain reaction (PCR) assay, fluorescence spectroscopy assay, a hybridization assay and sequencing.

79. The method of claim 68, wherein said relationship is determined by a sequence specific polynucleotide quantitation technique effected with a hybridization probe, a quantitative mass spectrometry based technique, or a quantitative polynucleotide amplification technique.

80. The method of claim 79, wherein said quantitative polynucleotide amplification technique comprises detection of labeled oligonucleotide probe binding or detection of dye binding.

81. The method of claim 68, wherein calculating said relationship further comprises the step of normalizing ratio values for the copy number of said first and said second distinct polynucleotide sequences.

82. The method of claim 68, wherein the determination of the linkage relationship between two or more distinct polynucleotides comprises the steps of:
a. obtaining a sample of tissue from a transgenic plant;
b. extracting DNA from the tissue sample;
c. digesting the DNA with a restriction enzyme
d. annealing the DNA with biotinylated oligonucleotides corresponding to at least one site within a known sequence or to at least one site within a known selectable or scoreable marker sequence;
e. adding streptavidin-coated magnetizable beads to the annealing reaction;
f. magnetizing the beads;
g. eluting the trapped DNA;
h. determining the PCR Cycle Threshold (CT) values for the trapped sequences;
i. comparing the CT values and calculating a difference of said values; and
j. determining the linkage relationship between said DNA sequences, wherein a polynucleotides lower difference value indicates that the two distinct polynucleotides are linked.

83. A method of sequencing an isolated polynucleotide molecule, said method comprising the steps of:
a. obtaining a sample comprising one or more polynucleotides;
b. isolating a first distinct polynucleotide within said sample, in a manner that is not effected in a gel matrix by electrophoresis;
c. amplifying said first distinct polynucleotide from step b), and
d. sequencing said first distinct polynucleotide.

84. The method of claim 83, wherein any of said polynucleotides are obtained from a transgenic plant selected from the group consisting of barley, corn, oat, sorghum, turf grass, sugarcane, wheat, alfalfa, banana, broccoli, bean, cabbage, canola, carrot, cassava, cauliflower, celery, citrus, cotton, a cucurbit, eucalyptus, flax, garlic, grape, onion, lettuce, pea, peanut, pepper, potato, poplar, pine, rye, rice, sunflower, safflower, soybean, strawberry, sugar beet, sweet potato, tobacco, tomato, ornamental, shrub, nut, millet, and pasture grass.

85. The method of claim 83, wherein said first distinct polynucleotide comprises a transgenic polynucleotide of agronomic interest or a polynucleotide that encodes or is operably linked to a selectable or scoreable marker gene.

86. The method of claim 83, wherein said isolation of said first distinct polynucleotide segment in step b) is effected in solution by a hybridization probe.

87. The method of claim 83, wherein any of said isolated distinct polynucleotide sequences is isolated by a method selected from the group consisting of: lysis, heating, alcohol precipitation, salt precipitation, organic extraction, solid phase extraction, silica gel membrane extraction, CsCl grandient purification, and any combinations thereof.

88. A method for identifying a transgenic plant containing a transgene insertion in an undesirable genomic location, comprising the step of identifying a transgenic plant wherein a transgene has inserted into a genomic region comprising one or more retrotransposon sequences, thereby identifying a transgenic plant containing a transgene insertion in an undesirable genomic location.

89. The method of claim 88, wherein said retrotransposon is a TY3/gypsy-like retrotransposon.

90. The method of claim 88, wherein said transgene insertion is adjacent to a retrotransposon or is within a retrotransposon.


SEQUENCE LISTING (7 sequences)

SEQ ID NO: 1 - DNA, 789 bp, Artificial
  T-DNA insertion in Arabidopsis genome
gccacaaatgaaaatcaaagcgaggggaaaagaatgtagagtgtgactacgattgcatgcatgtgatttaggtaattaagttacatgattgtctaattgtgtttatggaattgtatatagaacccagcggtactcgctgaggcgatcgcgggccctgatcacctgtcgtacagtatttctacatttgatgtgtgatttgtgaagaacatcaaacaaaacaagcactggctttaatatgatgataagtattatggtaattaattaattggcaaaaacaacaatgaagctaaaattttatttattgagccttgcggttaatttcttgtgatgatctttttttttattttctaattatatatagtttcctttgctttgaaatgctaaaggtttgagagagttatgctctttttttcttcctctttcttttttaactttatcatacaaattttgaataaaaatgtgagtacattgagctcatttaaataagcttgatggggacaagccatttaagacgaaagctatgaattaatgtgtcggcatcatatgaaactagcttgtagctagaaacgtttgaactttataaaatttatgatttatggatcgtgatcattgagaccgttgaagcaagacattggtatttttttggagctaaaacacaattttttgggtatacaacatcaacatatttatctttttcaaaaaatataaaaatatttgtgtagtcatattcatatagtattaaactcatgtcgagaagaaaaaggtttttatttgtatagtatactttta

SEQ ID NO: 2 - DNA, 1356 bp, Artificial
  T-DNA insertion in Arabidopsis genome
ttcaaacttcacacttataaattagtaaacatttgacgggtagactgttgcaaccaaggagaattgacggaaatggtcgtttattagcaatttagaacaaagattggccaattgttgactagtaagcttctcgagtggtggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaaggagaaagagaaagagggttcactttaatattgttgtcttttaattcaagtgttgcgttttatgtaagggttttgatacactctgtatttaaaggagggtttcccgtttagggcctcctcaatcattacctaccaaaaacattactattttctaatctctctctctttcctctccgccgcatctggcgcaatactatctccctcctatttcttcttgttacattctcgagtgaactgtcaagtttatcattttgattcttcgattttctttcttagtgccgctgatttagacgcaatttcagctagcttgcctacaaagtcatcaattttttcggtgtccttcatcttaaggttattaaattcgttcattagtgtctgtagtctagcttctttaatcgttatgcacctacgttcctagccttgatagcttcccaagctttctttgctaccttgaattctccaatctactaagttagagattcatgtatggattgaactaacaaggctatggccaagatcttatcttcatcgcttggatcggttgactctgtttcacttggagaacgatctgcatcttcatagcccaagctgtataattggtttgagtaagattgggatattgaatcgaagaagaccctccttcttttgatttcggtgttgacgcaactatttcactcatctctctctcttgatcttacaagttctccctatacggtcttaagatcttgtaagctctcataccaattcatgtgtttaaagtttgtatgtaagaacacaagatcaataagaatgactctcttattatgctttagaaacttacacaaaactaaagctttcaagttctctcaaatctcacaaactctcttaaggaccatgttacaactcaacatgtccttatatgtaaaagaatctagtcctaa

SEQ ID NO: 3 - DNA, 1214 bp, Artificial
  T-DNA insertion in Arabidopsis genome
attttctaattatatatagtttcctttgctttgaaatgctaaaggtttgagagagttatgctctttttttcttcctctttcttttttaactttatcatacaaattttgaataaaaatgtgagtacattgagctcatttaaataagcttgatggggatcagattgtcgtttcccgccttcagtttaaactatcagtgtttgattgagtataagaactaaaagagcaaccggatctaaaaccctaagtagtgtttccttgttagaagatacaaatccaaagactcatacggacttcatctacaccatcaaagctttgagaagcaagaagaggcttggttagtgttttggagtcgagtatgacttgatgtcatgtgtatgagggggtataaaaactagaaccgcaacagatcttcaaagcctaagtattgtatccttgttagaagatacaatgacaaacagtcatatgaacttcggctacaccatcaaagctttaagaagcaagaagaagcttggttagtgttttggagtgaaatatgactagatgtgatgtgtatgagtgagtataacaactagaaccgcaacaagattttaaaagcctaagtatgtatcttttacattgttcgtttgtctatagctggattcagcgaccaattatagcgacaatcgctaataactgtctctgtgttgttcgtttgactgtcgctataaacagtcgctatattgtttagaaaaattaattattttttgtgctaatttatattttaaaatcaaaatataaatcatatatatcatacaccatgagaaaaaataaattaatatatttctttattttagttttatcaaatttaatttgactataattagcttacattattttaatttaatttattttaacttcatttaataaaaaaattaataattaaactagaactcaaacccctcgtgatatcgcagagaaaaatattataaaagacacaagtgttttggagtcaaatatgactagatgtcatgtgtatgagtgagtataacaactaaaaccgcaaccgaatcttaaaagcctaagtattgtatcctggttagaagatataaagacaaatactcatacggacttcggctacaccatcaaagttttgagaagcaagaagaagcttgttagtgttttggagtcgaatatgacttgatatcatgtgtatgattgactaaaagaacttaaac

SEQ ID NO: 4 - DNA, 21 bp, artificial
  biotinylated oligonucleotide
cggaggattgctcgctcccga

SEQ ID NO: 5 - DNA, 21 bp, Artificial
  biotinylated oligonucleotide
ttcgtcgcagtccacgccgtt

SEQ ID NO: 6 - DNA, 22 bp, Artificial
  biotinylated oligonucleotide
gcttttgcgagctttgtggtgc

SEQ ID NO: 7 - DNA, 23 bp, Artificial
  biotinylated oligonucleotide
cgttttcagcccatccttcctcc